(19) United States
(12) Patent Application Publication
(10) Pub. No.: US 2014/0363379 A1
(43) Pub. Date: Dec. 11, 2014

(54) NUTRITIONAL APPROACH TO THE USE OF ERGOTHIONEINE AND VITAMIN D2 FOR HAIR, NAIL AND SKIN GROWTH

(71) Applicant: ENTIA BIOSCIENCES, INC., Sherwood, OR (US)

(72) Inventor: Marvin S. Hausman, Sherwood, OR (US)

(21) Appl. No.: 14/369,397

(22) PCT Filed: Dec. 21, 2012
(86) PCT No.: PCT/US12/71170

§ 371 Date: Jun. 27, 2014

Related U.S. Application Data
(60) Provisional application No. 61/581,480, filed on Dec. 29, 2011.

(51) Int. Cl. A61K 36/06; A61K 31/4172; A61K 31/592; A61K 31/593; G01N 33/68; A61K 8/49; A61K 8/67; A61Q 3/00; A61Q 19/00; A61Q 7/00; A61K 35/74; A61K 8/99

(52) U.S. Cl. CPC A61K 36/06; A61K 35/748; A61K 31/4172; A61K 31/592; A61K 31/593; A61K 8/99; A61K 8/4946; A61K 8/67; A61Q 3/00; A61Q 19/00; A61Q 7/00; G01N 33/689; G01N 2800/20
USPC 424/9.1; 424/195.15; 424/195.16; 514/167; 435/7.1

(57) ABSTRACT

Nutritional products, including food and/or beverage compositions, pharmaceutical and cosmetic preparations and methods of use are disclosed for the prevention, suppression and treatment of skin, hair, and nail conditions for the improvement of skin, hair and nail growth through the identification and/or use of stem cells, as well as maintenance of healthy tissues. Uses of Ergothioneine and/or Vitamin D to neutralize free radicals, and improve skin, hair and nail growth are disclosed.

### CROSS-REFERENCE TO RELATED APPLICATIONS

This application claims priority under 35 U.S.C. §119 to provisional application Ser. No. 61/581,480 filed Dec. 29, 2011, herein incorporated by reference in its entirety.

### FIELD OF THE INVENTION

This invention relates to whole foods, extracted ingredients, compositions, including nutritional products and nutritional foods for improving hair, nail and skin growth and/or health and treating comorbid conditions by the use of Ergothioneine and Vitamin D2. In an aspect, the extraction of Ergothioneine and Vitamin D2 from whole food sources and/or bacterium for use in nutritional products, medical foods and other treatments relating to hair, nail and/or skin growth are also disclosed.

### BACKGROUND OF THE INVENTION

Human hair typically grows at a rate of around half an inch (1.25 centimeters or 12.5 millimeters) per month, and this rate of growth is consistent across the head. Average fingernail growth rate in a healthy person is 0.347 centimeters or 3.47 millimeters per month. In addition skin cells are constantly replenishing to replace dead cells throughout the body, in particular on exterior portions of the body, such as face, arms, legs and torso. Several factors can influence both the rate and quality of hair, nail and/or skin growth, including diet, age, and general health.

Scientists have also suggested that control of free radicals and chronic inflammation may also play a protective role in skin, hair and nail health. These opinions are based on the fact that rapidly dividing regenerative stem cells in various tissues, including skin, hair and nails are particularly sensitive to free radical damage. For example, it has been shown that oxidative stress and free radical damage is a basis for hair loss (Hoffmann, J. Investigative Derm. Symposium Proceedings, (2004) 4, 235-238). Cytokines are also demonstrated to cause hair loss, cellular damage, and premature graying (Trueb, Int. J. Trichology, (2009) 1(1): 6-14). Research has demonstrated a dose dependent inhibition of TNF-alpha free radical mediated reactions with the use of the antioxidant L-Ergothioneine, indicating a potential strategy for use of ergothioneine derived from food plants to benefit chronic immunodeficiency diseases (Xiao et al., Biofactors (2006), 27 (1-4), 157-65).

Tumor Necrosis factor alpha (TNF-a) is a type of cytokine that causes the immune system to attack healthy tissues in the body. For example, elevated TNF-a can cause a repetitive inflammatory cascade and result in programmed cell death (apoptosis) of hair cells; a condition called androgenetic alopecia (Rucker et al., Br. J. Dermatol., (2000), 143 (5), 1036-1039). There are currently few TNF-alpha drug inhibitors on the market, such as Enbrel (etanercept) which is referred to as a biologic response modifier or a recombinant human soluble tumor necrosis factor receptor fusion protein. Enbrel is genetically engineered to be like the receptors in the body which bind to tumor necrosis factor (TNF-alpha). Enbrel works by soaking up excess TNF before it can attach to natural receptors and has proven effective in treatment of moderate to severe rheumatoid arthritis, moderate to severe juvenile rheumatoid arthritis, psoriatic arthritis, ankylosing spondylitis, and chronic moderate to severe cases of the dermatologic condition plaque psoriasis. However, such a biologic response modifier is too severe treatment regimen to consider for use in treating hair loss and/or decreased hair, nail and/or skin growth. Therefore, there remains a need for treatment regimens for milder early occurrences of these conditions which have a causal link to cytokine activity in body tissues.

There are additional identified links between health conditions and decreased hair growth and/or hair loss and/or skin disorders. For example, a high incidence of insulin resistance (IR) among people with male and female pattern hair loss, namely Alopecia Areata, has been identified (Karadaq et al., Cutan. Ocul. Toxicol., 2012 Aug. 24 (online publication)). A further link between the increased inflammatory cytokines in the IR patients and the hypothalamic-pituitary-adrenal axis activation was identified as a causal factor of the hair loss. This causal link is significant as serum levels of Vitamin D have been shown as deficient in IR patients, such that Vitamin D deficiency has been implicated as a risk factor for glucose intolerance and may play a role in the regulation of insulin production. Maqhbooli et al. showed a positive correlation between 25-hydroxyvitamin D concentrations with insulin sensitivity in pregnant women, indicating that vitamin D deficiency is a potential confirmative sign of insulin resistance (Diabetes Metab. Res. Rev., 2008; 24(1):27-32). Further support was identified in a correlation between low vitamin D levels and low adiponectin levels, obesity and insulin resistance (Nunlee-Bland et al., J. Pediatr. Endocrinol. Metab., 2011; 24(1-2):29-33). Further studies showed beneficial effects of vitamin D supplementation, wherein bringing 25(OH)D levels above 80 nmol/L improved insulin resistance by 12% and increasing vitamin D levels from 25 to 75 nmol/L resulted in a 60% improvement in insulin sensitivity (Grant et al., Mol. Nutr. Food Res., 2010, 54, 1-10). The studies suggest a strong role for the supplementation and/or treatment with the compositions disclosed in the present invention.

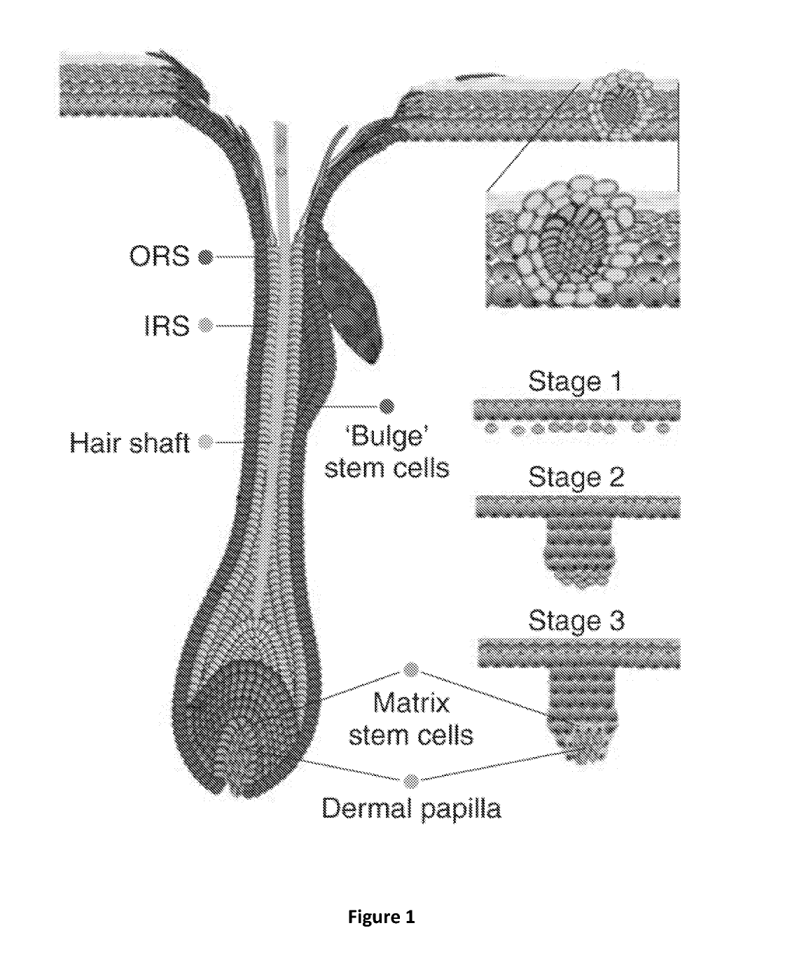

In addition to the various connections between unhealthy skin, hair and/or nail growth and oxidative stress, immune system activity, maladaptive response and/or dysfunction, including release of cytokine-type signaling molecules and/or vitamin D deficiency, the tissue structure of skin, hair and nail cells is also important. Hair follicles and nail matrix are small complex organs capable of repetitive active growth. The interaction and cross-talk between dermal fibroblasts and epidermal cells is critical for hair follicle formation and cycling. There are also dermal and epidermal stem cell niches that are activated to rebuild the hair follicle, regenerate cells and treat pattern hair loss. FIG. 1 shows the anatomy of a dermal papilla and hair shaft, including matrix stem cells and bulge stem cells. In the nail, the germinal matrix is responsible for the production of the cells that become the nail plate. In contrast to the stem cells in hair follicles, the localizations of stem cells in nails have been less characterized. However, recent studies by Nakamura and Ishikawa utilizing a radioactive label showed that nail stem cells appear to be dermal fibroblasts and reside in the basal layer of the nail matrix adjacent to the nail bed (Nakamura & Ishikawa, J. Inves. Derm. (2008) 128, 728-730).

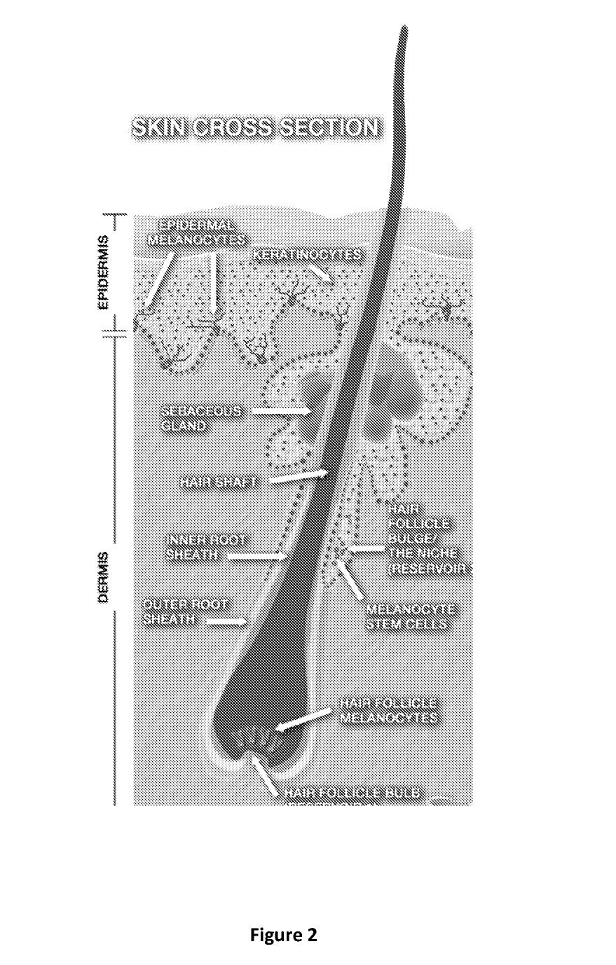

The anatomic and histologic location of melanocytes and a cross sectional view of skin is shown in FIG. 2. It is known that hair and skin color comes from a protein called melanin. Melanocytes are melanin-producing cells located in the bottom layer (stratum basale) of the skin's epidermis and melanin is the pigment primarily responsible for skin, hair and eye color. Recent findings suggest that the melanocytes also come from stem cells that are in close proximity to the hair bulb. These stem cells are often called “bulge or niche” stem cells and they migrate down to the hair bulb to be incorporated within the hair shaft to be formed. Gray hair happens when the hair loses its melanocytes. Aging and gray hairs occur when the stem cells are no longer able to replace melanocytes as they did at a younger age. There is further evidence of oxidative stress-induced loss of melanocytes from the human hair follicle during aging (Arck et al., FASEB (2006), 20:9, 1567-1569). In addition, a higher frequency of oxidative stress associated with mitochondrial DNA damage occurs in graying hair follicles. This is significant as oxidative stress and free radicals are generated by a multitude of environmental and endogenous challenges, such as for example, radiation, inflammation, emotional stress, and even early aging.

An additional factor playing an important role in cell function, namely stem cell function, including cell maintenance and aging, is telomere length and telomerase (reverse transcriptase) activity. Telomeres, guanine-rich tandem DNA repeats of the chromosomal end, provide chromosomal stability, and cellular replication causes their loss. It has been shown that in somatic cells, the activity of telomerase is usually diminished after birth resulting in telomere length that is gradually shortened with cell divisions, triggering cellular senescence (Hiyama & Hiyama, Brit. J. Cancer, 2007; 96: 1020-1024). Notably, the level of telomerase is low in most human stem cells, whereas it is upregulated in cells that undergo rapid expansion, such as keratinocytes (Haik et al., Oncogene, 2000, 19:2957-2966). It is known that keratinocytes are produced by the stratum spinosum skin layer of the epidermis and as such play an important role in regeneration of old and dying skin cells. Therefore, the finding of overexpression of the Ergothioneine Transporter in the stratum spinosum strongly suggests an important role for Ergothioneine in the regeneration, proliferation, and repair of diseased and aging skin, as disclosed herein the present invention.

Injured skin is known to generate an electrical signal to promote healing. The development of a net charge or potential is called a zeta potential Skin cells respond to the zeta potential as part of the healing process. Early adherence of a skin substitute to a wound surface is paramount if it is to function as a skin equivalent. (Morykwas, Plast. Reconstr. Sung., (1987) 79:732-9). Ergothioneine is a potent electron donor and as such can cause biochemical modifications of natural and synthetic skin to increase the zeta potential and aid in skin healing as well a skin substitute adherence as part of surgical repair. The application of this technology using Ergothioneine with and without Vitamin D can mimic these natural healing signals and aid in tissue healing and rejuvenation. Ergothioneine, a potent natural antioxidant aids the body in maintaining an endogenous electric field profile for skin healing and rejuvenation.

The complex anatomy of skin, hair and nail cells demonstrates dynamic cellular systems. The various skin, hair and nail cells are also known to have certain receptors expressed in the cells and tissues throughout the body, which are now being realized to play an important role in the maintenance and growth of healthy cells. For example, the Vitamin D receptor (VDR) is expressed in numerous cells and tissues throughout the body, including the skin. The hair follicle is formed by reciprocal interactions between an epidermal placode, which gives rise to the hair follicle keratinocytes and the underlying mesoderm which gives rise to the dermal papilla. Vitamin D and its receptor (VDR) have been shown to have a number of effects on cutaneous homeostasis (Bikle & Pillai, Endocr. Rev., 4(1): (1993) 3-19). A super-active form of Vitamin D (VD3) has been identified as having benefits in boosting stem cells to enhance and maintain their ability to induce hair growth. It has been reported that dermal papilla cells (DPCs) can stimulate epithelial stem cells to become hair and that VD3 increases the transforming growth factor TGF-β2 and alkali-phosphatase activity, which are both essential features of hair-inducing DPCs (Aoi et al., Stem Cells Trans. Med., (2012) 1:615-626). The newly identified therapeutic potency of VD3 on hair regeneration shows a link between Vitamin D and the Vitamin D receptor with human DPCs and hair growth, including providing a potential treatment mechanism for hair transplantation. The limitation of such research is the narrow focus on activating the vitamin D receptor to initiate hair growth. The present invention provides additional Vitamin D sources and its analogues including Vitamin D2 (i.e. beyond solely Vitamin D3) to afford the unexpected benefits for hair, nails and skin.

Further benefits of Vitamin D include its pleiotropic effects beyond its traditional role in calcium homeostasis. There are hundreds of genes with vitamin D receptors with response elements directly or indirectly influencing cell cycling and proliferation, differentiation, and apoptosis (Samuel & Sitrin, Nutrition Reviews, 2008, 10; 1753-4887). Vitamin D compounds also affect cell functions that are nongenomic. For example, the noncalcemic actions of vitamin D influence normal and pathological cell growth, carcinogenesis, immune function, and cardiovascular physiology. Specifically, the active metabolite of Vitamin D, 1,25-dihydroxyvitamin D, has been shown to have pro-differentiation and antiproliferative effects on keratinocytes that are mediated by interactions with its nuclear receptor. These and other reports of the vitamin D receptor in skin and hair and responsive stem cells within these tissues support the need for higher levels of Vitamin D to correct deficiencies and/or to supplement and treat with the administration of Vitamin D sources (J. Steroid Biochem. Mol. Biol., 2010; 121(1-2):314-6).

In addition to the recognized benefits of Vitamin D3 in skin, hair and nail cells, the role of ergothioneine is further being identified. A unique Ergothioneine Transporter (ETT) has been identified in human cells with the gene, SLC22A4 coding for an integral membrane protein, OCTN1, and the key substrate of this transporter is L-Ergothioneine (ET) (Gründemann, Proc. Natl. Acad. Science, 102:14, 5256-5261 (2005). The ETT is described in further detail in PCT/EP2005/005613 and U.S. patent application Ser. No. 11/569,451, entitled “Identification of Ergothioneine Transporter and Therapeutic Uses Thereof,” the entire contents of which is herein incorporated by reference. SLC22A4 is a sodium-ion dependent transporter that efficiently and specifically carries L-Ergothioneine across the cell membrane. The ergothioneine transporter has been described as a powerful and highly specific uptake transporter; serving to effectively charge cells with as much ET as is available. In addition, ETT is the first and so far only biomarker of ET activity—only cells with strong expression of ETT can accumulate ET to high levels. As a result, in the human body, the ability to absorb, distribute, and retain ET depends entirely on this specific transporter. Moreover, its existence implies a beneficial role for ET and its blockade or inactivation is expected to be an important model for animal and/or human studies to better understand the function of ET (Gründemann, Prev. Med., 2012 May; 54:S71-4).

ETT was identified as the first molecular marker of Ergothioneine activity proving to be necessary for the supply of ET. Ergothioneine is a naturally occurring amino acid and thiourea derivative of histidine and is a very potent antioxidant and scavenger of free radicals and has the ability to protect against oxidative skin damage. Further studies have shown the presence of a genetically endowed transporter, OCTN-1 in skin cells that appeared to have the ability to recognize and transport and utilize the amino acid L-Ergothioneine (ET) (Dong et al., J. Cosmet. Dermatol., (2007) 6(3):183-8). The identification of ergothioneine transports is significant as studies at Johns Hopkins School of Medicine have shown that the antioxidant ergothioneine protect cells against radiation and damage to cellular DNA (Paul & Snyder, Cell Death and Differentiation, (2009) 1-7). The high density of ET within mitochondria implies a unique role in protecting mitochondrial DNA from damage induced by free radicals and reactive oxygen species. Mitochondria are cytoplasmic organelles responsible for life and death. Evidence from animal and clinical studies suggest that mitochondria play a critical role in aging, cancer, diabetes and neurodegenerative diseases such as Alzheimer's disease and Parkinson's disease (Simon et al., Neurobiol. Aging, (2004) 25, 71-81; Lin et al., Nature, (2006) 443:787-795; Reddy, CNS Spectr., (2009) 14(8), 8-18).

The ETT has further been demonstrated as necessary for the supply of ET to erythrocyte (red blood cell) progenitor cells and to monocytes (white blood cells). Still further, the function of ET and its transporter (ETT) and its role in metabolism and the pathophysiology of human disease remain to be fully understood, but variations in SLC22A4 have been associated with susceptibility to inflammatory disorders, such as rheumatoid arthritis and Crohn's disease, and expression has been documented in a variety of human tissues, including inflammatory cell types such as macrophages and monocytes. The existence of a specific ET transporter suggests that ET is advantageous to long-term human health (Grigat et al., Biochem. Pharmacol., (2007) 74, 309-316). Additional research at Johns Hopkins University School of Medicine has suggested that ET is as potent as glutathione. The dietary origin of ET along with toxicity associated with its depletion, as well as the research showing antioxidant cytoprotective activity, strongly supports its importance to human and animal health.

Further supportive evidence of the potential protective role of ET was recently shown by the presence of elevated levels of ET in the red blood cells of pregnant women with the condition preeclampsia (a condition having no known cause). The symptoms of preeclampsia include high blood pressure, protein in urine and fluid retention and affects almost 10% of pregnancies after 20 weeks. Left untreated, the condition can cause a range of problems such as growth restriction in babies and even fetal and maternal mortality. Ergothioneine having known antioxidant properties has been proposed as a treatment for reducing the risk of preeclampsia; however confounding to researchers is that ET has been found to be in excess for women with the condition.

Ergothioneine is a unique, naturally occurring, very stable antioxidant that cannot be made in human cells and must be absorbed from the diet; It is found in most plants and animals, but highly concentrated in mushrooms. A human bioavailability study was conducted in the Department of Food Science, Pennsylvania State University (Weigand-Heller, Prev. Med., 2012: 54; S75-78), wherein a postprandial time course study of varying mushroom doses (0 g, 8 g, and 16 g) was used to evaluate the bioavailability of L-Ergothioneine (ET) from mushrooms. This study convincingly indicated that L-Ergothioneine is bioavailable in humans through the consumption of mushrooms (peak of ET appeared in red blood cells (RBC) after only 2 hours of mushroom consumption). The appearance of ET within red blood cells in such a short time after ingestion of mushrooms strongly suggests that human tissues and cells contain an active mechanism of transport for ET.

In addition to dietary studies of ET from mushrooms, the work of Kalaras and Beelman both identified and quantified the ergosterol-derived photoproducts previtamin D2, lumisterol 2 and tachysterol 2 in white button mushrooms (Agaricus bisporus) following treatment with pulsed UV (PUV) light (Food Chemistry Volume 135, Issue 2, 15 Nov. 2012, 396-401). Mushrooms were treated with up to 60 pulses of PUV irradiation and the formation of major photoproducts was observed to increase as a function of dose. Vitamin D2 was the most abundant product, followed by previtamin D2, lumisterol 2 and tachysterol 2 in order of decreasing abundance, whereas untreated mushroom samples were not observed to contain detectable levels of any photoproduct, demonstrating for the first time the production of these photoproducts in UV irradiated mushrooms. Other valuable health benefits of mushrooms are disclosed in U.S. patent application Ser. No. 12/887,276, titled “Vitamin D2 Enriched Mushrooms and Fungi for Treatment of Oxidative Stress, Alzheimer's Disease and Associated Disease States,” and Ser. No. 12/386,810, titled “Methods and Compositions for Improving the Nutritional Content of Mushrooms and Fungi,” which are herein incorporated by reference in their entirety.

Mushrooms are a valuable health food—low in calories, high in vegetable proteins, chitin, iron, zinc, fiber, essential amino acids, vitamins and minerals. They are also an excellent source of organic selenium compounds, riboflavin, pantothenic acid, copper, niacin, potassium and phosphorous. Selenium is needed for the proper function of the antioxidant system, which works to reduce the levels of damaging free radicals in the body. Selenium is a necessary cofactor of one of the body's most important internally produced antioxidants, glutathione peroxidase, and also works with vitamin E in numerous vital antioxidant systems throughout the body. Mushrooms are also a primary source of natural Vitamin D, in the form of D2, which is naturally present in very few foods. Most other natural food sources of Vitamin D, in the form Vitamin D3, are of animal, poultry or seafood origin.

Vitamin D is a fat-soluble vitamin that is naturally present in very few foods, added to others, and available as a dietary supplement. Vitamin D comes in two forms (D2 and D3) which differ chemically in their side chains. These structural differences alter their binding to the carrier protein Vitamin D binding protein (DBP) and their metabolism, but in general the biologic activity of their active metabolites is comparable. It is also produced endogenously when ultraviolet rays from sunlight strike the skin and trigger Vitamin D synthesis. So one must either ingest Vitamin D or sit in the sun and soak up UV rays, so that it may be synthesized endogenously. Most of the population is deficient in Vitamin D. The risks of sun exposure continue to gain attention, including the association of sun exposure with pre-cancerous (actinic keratosis) and cancerous (basal cell carcinoma, squamous cell carcinoma and melanoma) skin lesions—caused by loss of the skin's immune function, fine and coarse wrinkling of the skin, freckles, discoloration of the skin, and Elastosis—the destruction of the elastic tissue causing lines and wrinkles is well documented. Thus as people become more sensitive to the dangers of UV exposure, other dietary sources of Vitamin D become increasingly important for maintaining health.

There are two basic types of Vitamin D. Ergosterol is the basic building block of Vitamin D in plants and fungi. Cholesterol is the basic building block of Vitamin D in humans. When ultraviolet light from the sun hits the leaf of a plant or fungal tissue, ergosterol is converted into ergocalciferol, or Vitamin D2. In just the same way, when ultraviolet light hits the cells of our skin, one form of cholesterol found in our skin cells—called 7-dehydrocholesterol—can be converted into cholecalciferol, a form of Vitamin D3. The liver and other tissues metabolize Vitamin D, whether from the skin or oral ingestion, to 25OHD, the principal circulating form of Vitamin D, by the enzyme CYP27B1, the 25OHD-lahydroxylase. 25OHD is then further metabolized to 1,25(OH) 2D principally in the kidney, although other tissues such as epidermal keratinocytes and macrophages contain this enzymatic activity. 1,25(OH) 2D is the principal hormonal form of Vitamin D, responsible for most of its biologic actions.

Vitamin D has many roles in human health, including modulation of neuromuscular and immune function, reduction of inflammation, maintaining blood levels of phosphorus and calcium, promotion of bone mineralization and calcium absorption, maintaining a healthy immune system, and regulating cell differentiation and growth. Recent studies have also shown a link between vitamin D deficiency and diseases such as cancer, chronic heart disease, inflammatory bowel disease and even mental illness. In addition, many genes encoding proteins that regulate cell proliferation, differentiation, and apoptosis are modulated in part by Vitamin D. Many laboratory-cultured human cells have Vitamin D receptors and some convert 25(OH)D to 1,25(OH)₂.D. It remains to be determined what cells, tissues, and organs in the human body contain either D2, D3, or both vitamin receptors and what additional cells with Vitamin D receptors in the intact human can carry out this conversion from 25(OH)D to 1,25(OH)₂D.

It is an object of the present invention to provide a natural, cost effective method to promote hair, nail and skin growth in humans and animals.

It is an object of the present invention to control oxidative stress, free radicals and/or inflammation to provide a natural, cost effective method to protect skin, hair and nail health in humans and animals.

It is the object of the present invention to provide a natural, cost effective method to prevent loss of melanocytes and/or premature graying.

It is the object of the present invention to protect hair, nail and skin stem cells which are highly susceptible to oxidative damage (lipid peroxidation) caused by reactive oxygen species.

It is the object of the present invention to utilize a specific antibody to the SLC22A gene and messenger RNA for targeted nutritional therapy for stem cells essential to hair, nail and skin growth.

It is the object of the present invention to protect the health, function and viability of stem cells by supplying a source of ergothioneine and Vitamin D2, such as ErgoD2™ which maintains telomere length.

It is a further object of the invention to provide a composition and/or serum composition to supplement dietary sources of natural ergothioneine and Vitamin D2, such as Ergo-D2™, a potent anti-oxidant, anti-free radical, anti-inflammatory nutritional product, to stimulate and/or restore hair and nail growth, maintain skin health, and delay onset of age related and/or stress related grey hairs.

It is a further object of the invention to provide a composition, such as Ergo-D2™, to supply nutrients that can only be supplied in the diet for normal stem cell differentiation and proliferation within hair, nail and skin.

A further object of the present invention is to provide a dietary supplement or other food or beverage products which are high in nutritional values, particularly Vitamin D2 and Ergothioneine that is extracted from natural whole food sources and/or bacterial sources.

It is another object of the invention to provide dietary supplements, dietary ingredients or other food or beverage products obtained from whole, natural sources (such as Spirulina or oats) for use in treatment of patients in need of improved or restored hair and/or nail growth.

These and other objects of the present invention will become apparent from the description of the invention which follows.

### SUMMARY OF THE INVENTION

Methods for prevention of hair loss and/or nail deterioration, treatment for the same and related conditions and/or restored or improved skin, hair and nail growth are provided according to the invention. According to an embodiment, the invention creates an improved food or supplement product with a naturally enriched Vitamin D and Ergothioneine nutritional profile for the prevention, treatment and/or restoration methods disclosed herein for human skin, hair and nail growth, maintenance of natural hair colors, and normal skin cell growth with proper development of protective epidermal skin barriers. The products and methods of using the products according to the invention may be obtained from a variety of whole natural sources, including mushrooms, yeast, oats or barley or cyanobacteria, including Spirulina. Synthetic and natural dietary extracted Ergothioneine may be combined with phytonutrients, Vitamin D (including from enriched mushrooms and/or substrates (namely a mushroom or other fungi, and/or mycelia having enhanced content of Vitamin D or its analogs or derivatives)), beta and/or chitin glucans.

In an embodiment, the combination of Ergothioneine and Vitamin D improves the growth of hair and nails, and improves the structure and barrier protection of skin, providing significant clinical benefits for patients in need thereof. The compositions and products, as well as the methods of using the compositions and products according to the invention, may be provided as a daily supplementation regimen for prevention, restoration and/or as treatment regimens.

In a further embodiment, the inventions includes pharmaceutical and/or cosmetic compositions for the prevention of hair loss and/or nail deterioration, treatments for improved hair and nail growth, and improving the structure and barrier protection of skin. Such treatments for these conditions and related conditions are provided according to the invention.

### DETAILED DESCRIPTION OF THE FIGURES

FIG. 1 shows the anatomy of the hair bulb including the location of stem cells, and the structure of the dermal papilla and hair shaft, as is known by those of skill in the art.

FIG. 2 shows a cross section of skin, including the melanocyte stem cells within the hair bulb along with “the niche” melanocyte stem cells, as is known by those of skill in the art.

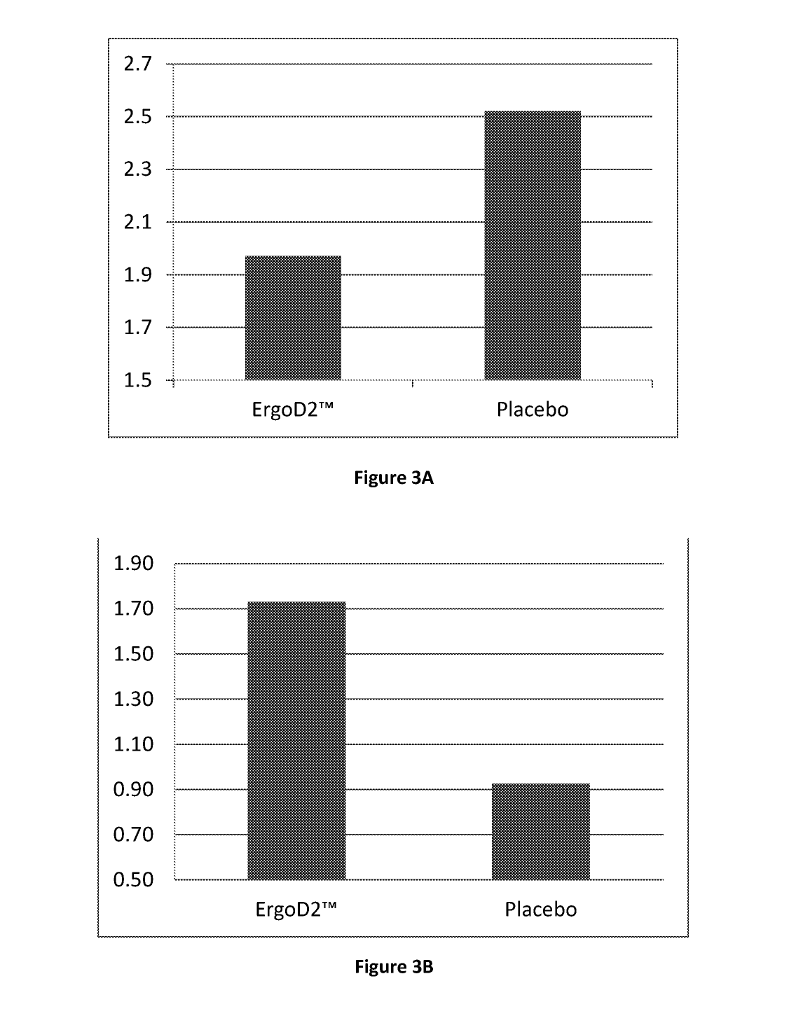

FIGS. 3A-B show graphs depicting alpha-synuclein concentration in treated and control groups according to embodiments of the invention.

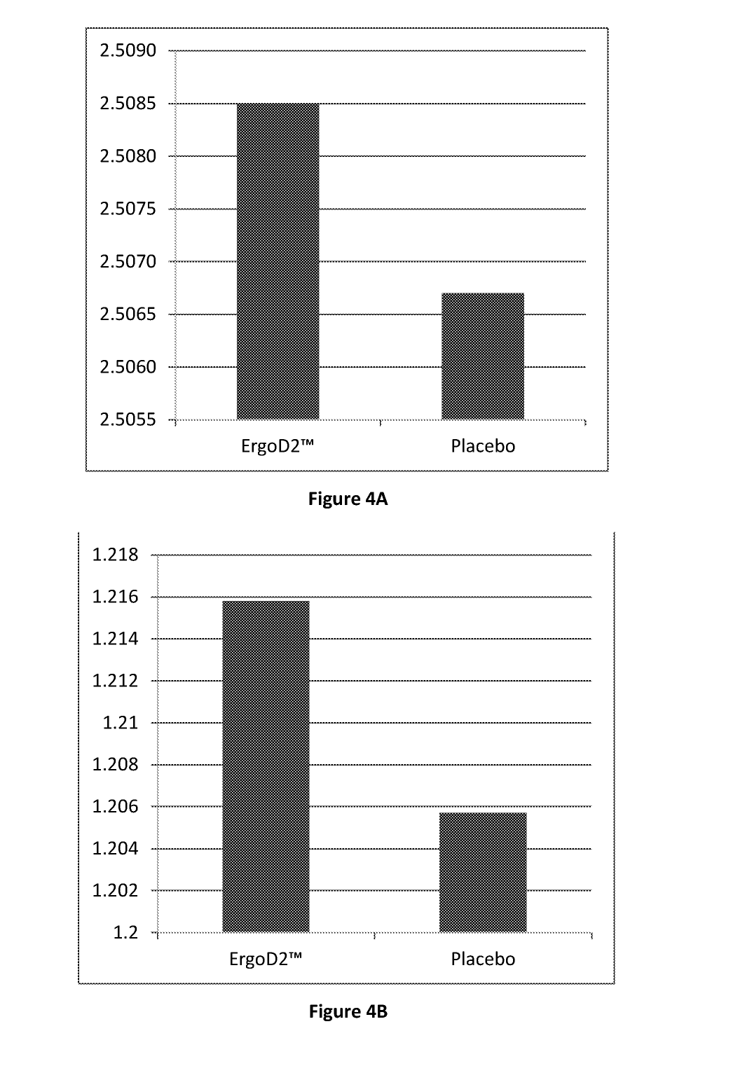

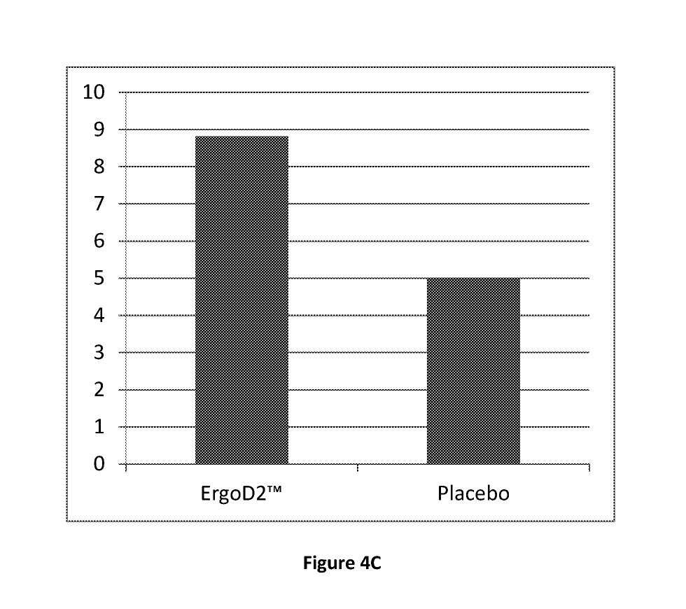

FIGS. 4A-C show graphs depicting that according to embodiments of the invention untreated transgenic Parkinson's disease (PD) mice had significantly lower glutathione concentrations than the PD mice treated with ErgoD2.

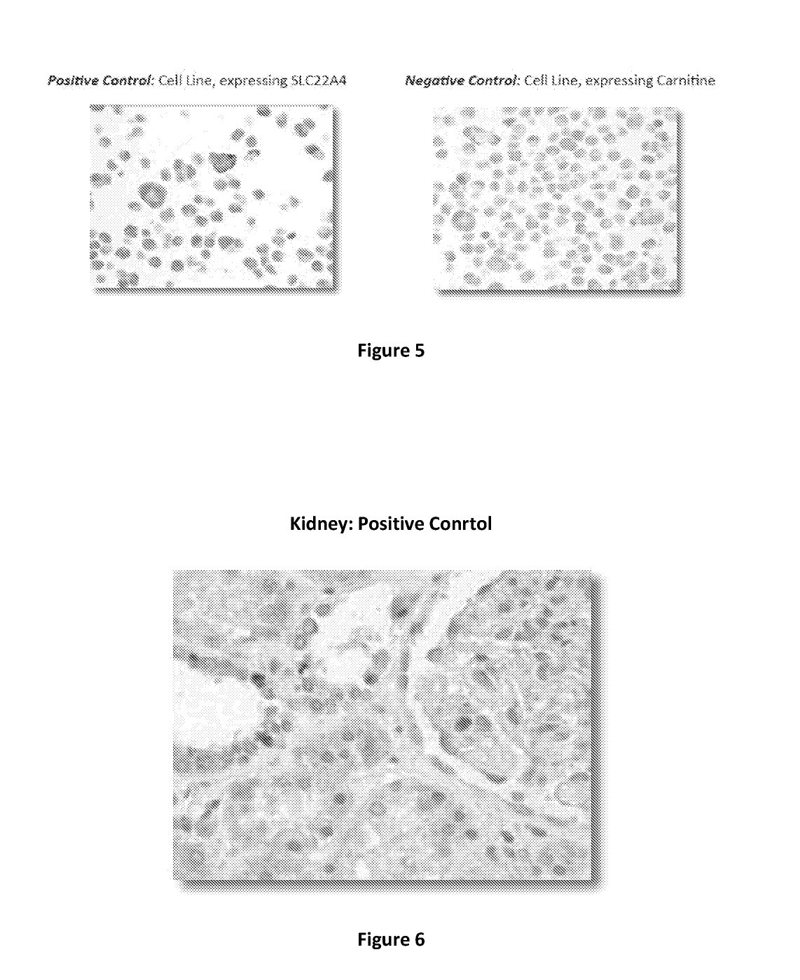

FIG. 5 shows a positive control cell line expressing SLC22A4, and a negative control cell line expressing carnitine.

FIG. 6 shows the proximal convoluted tubules (PCT) stained, indicating the reabsorption of ergothioneine in the human body via the PCT.

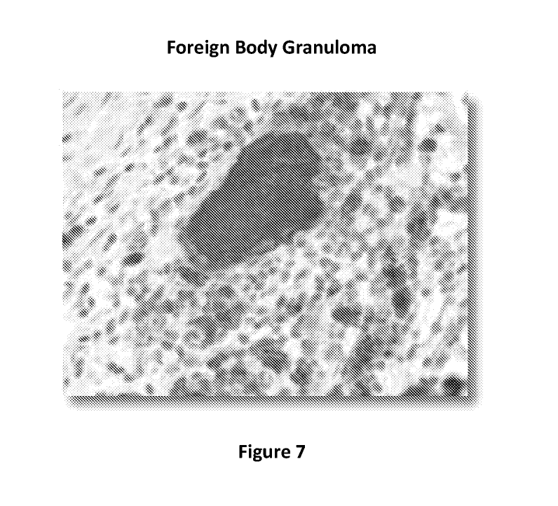

FIG. 7 shows the heavy staining of macrophages in the foreign body granuloma.

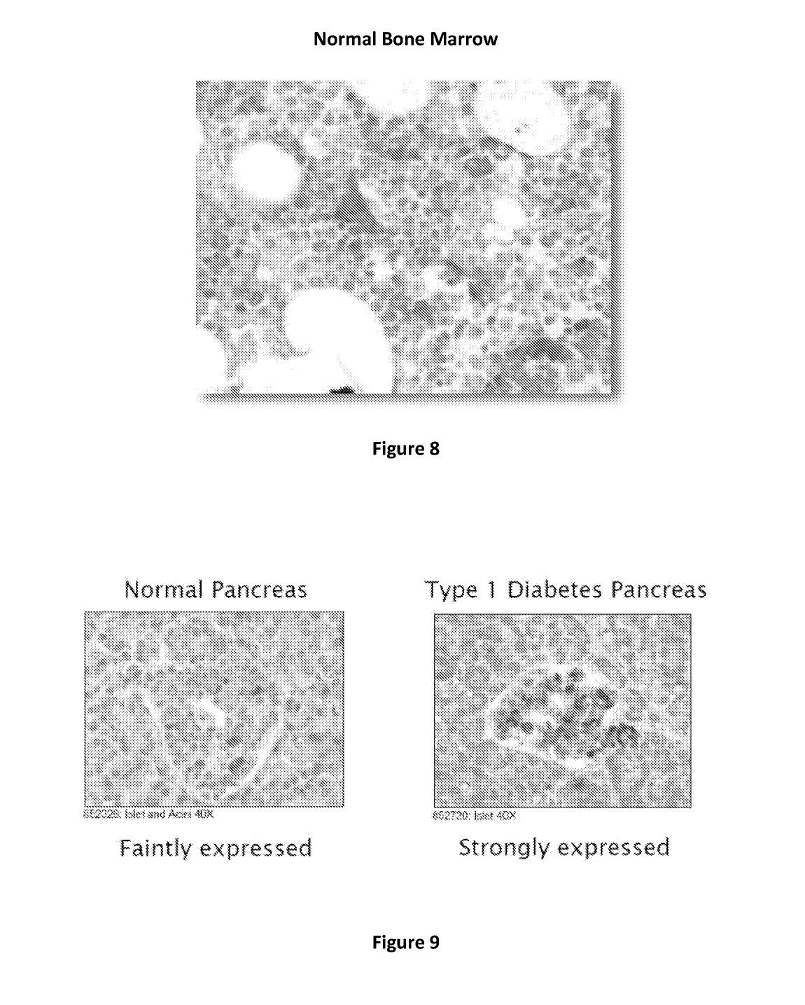

FIG. 8 shows the moderate to strong staining of macrophages, and the faint to moderate staining of megakaryocytes in the normal bone marrow of a patient.

FIG. 9 shows a photograph of pancreatic tissue samples and specifically of the Islet of Langerhans where insulin is produced. The SLC22A4 transporter protein (shown by the antibody containing vector red) is significantly increased in Type 1 diabetic tissue as compared to normal tissue.

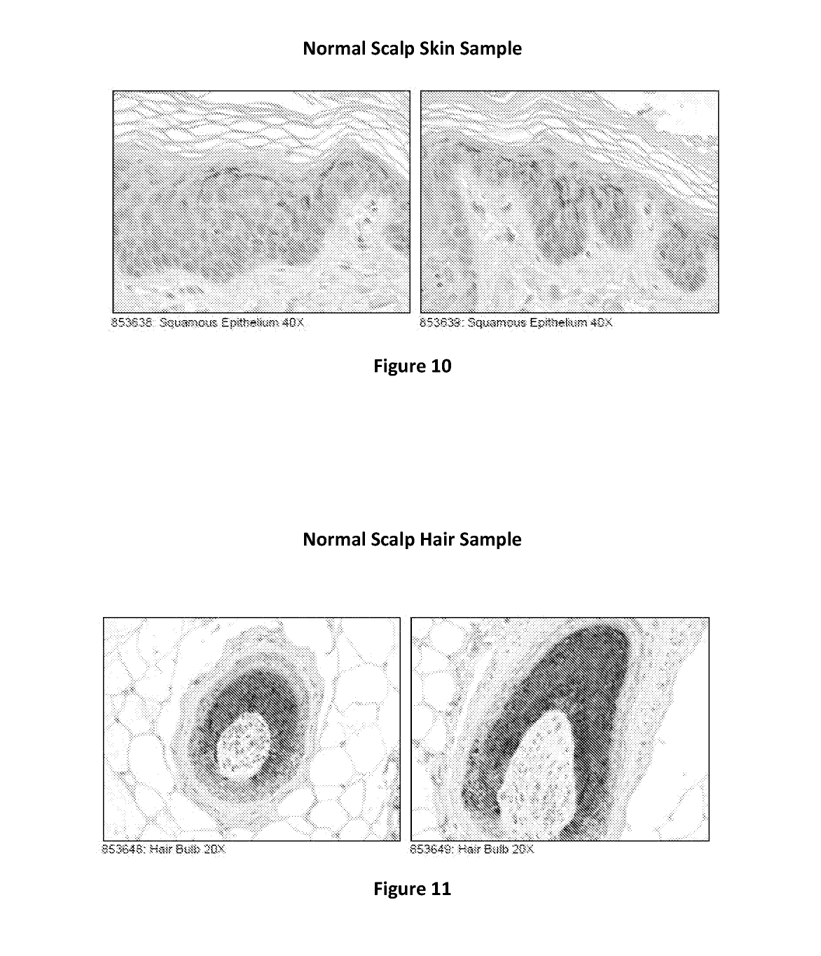

FIGS. 10-14 show photographs of scalp skin and hair samples from patients having normal skin conditions.

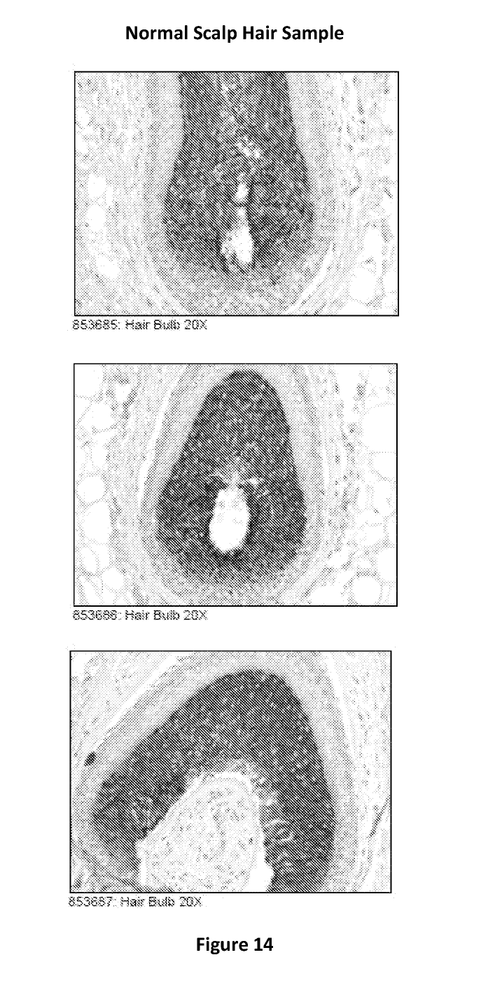

FIG. 14 shows dark colored melanin granules within the melanogenic zone of a hair sample and the hair follicle bulb cells with antibodies attached thereto, demonstrating the ETT within the hair follicle stem cells in humans.

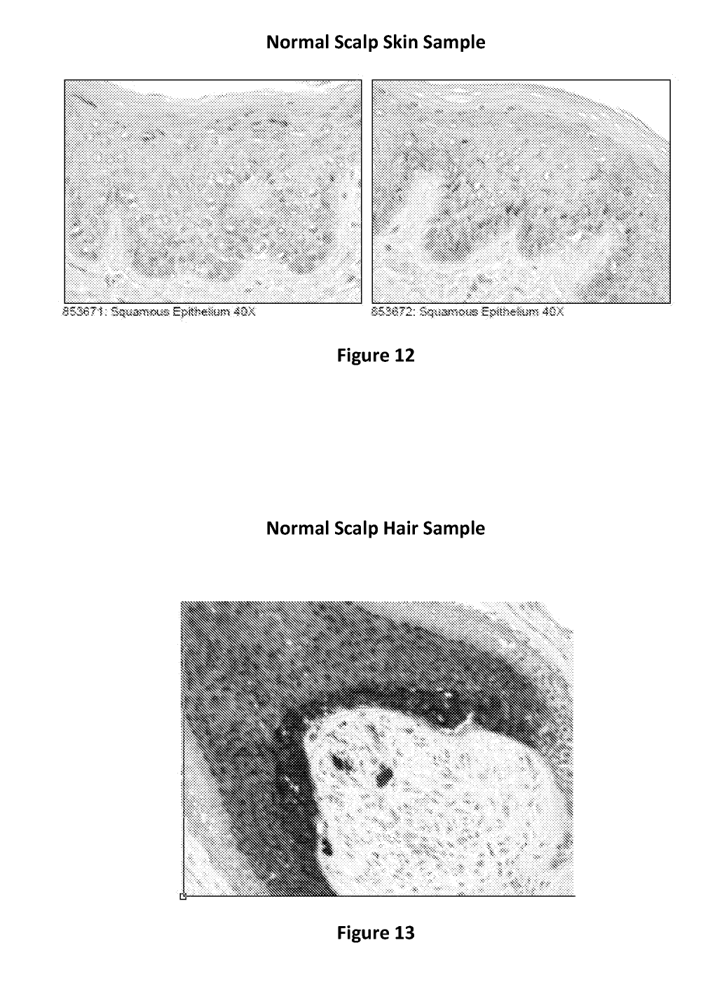

FIG. 12 show photographs of scalp skin and hair samples from patients having normal skin conditions.

FIGS. 13-14 show additional photographs of the hair bulb from hair samples of a patient having normal skin conditions.

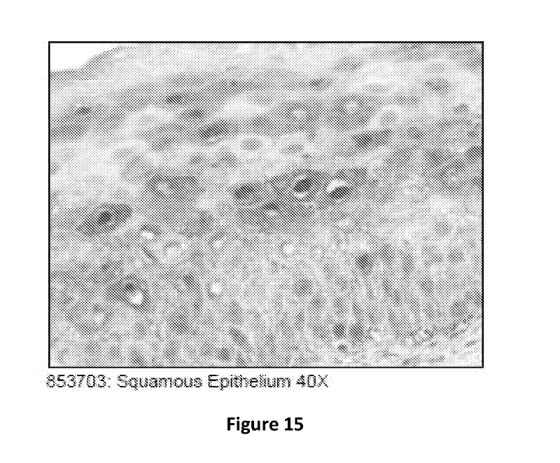

FIG. 15 shows a photograph of epithelium cells from a patient with alopecia (hair loss), showing strong nuclear staining of the stratum basalis.

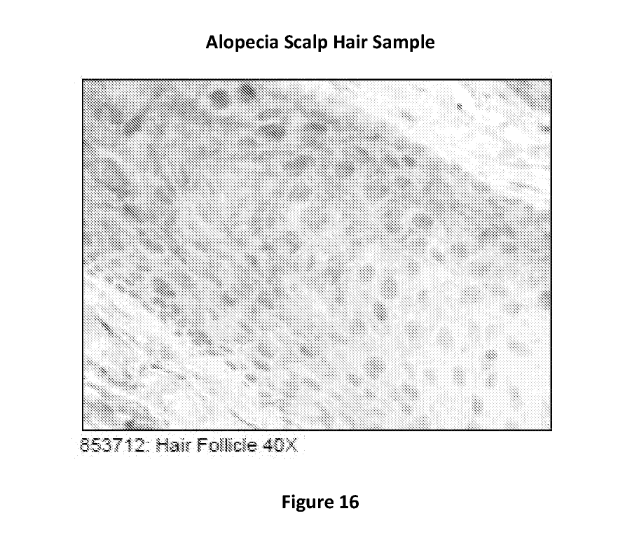

FIG. 16 shows a photograph of a hair follicle sample from a patient with alopecia (hair loss).

Various embodiments of the present invention will be described in detail with reference to the drawings, wherein like reference numerals represent like parts throughout the several views. Reference to various embodiments does not limit the scope of the invention. Figures represented herein are not limitations to the various embodiments according to the invention and are presented for exemplary illustration of the invention.

### DETAILED DESCRIPTION OF THE INVENTION

The embodiments of this invention are not limited to particular embodiments for compositions and uses of Ergothioneine and Vitamin D for skin, hair and/or nail growth, and/or maintenance and viability of hair, nail and skin cells, including hair, nail and skin stem cells, which can vary and are understood by skilled artisans. It is further to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form. Numeric ranges recited within the specification are inclusive of the numbers defining the range and include each integer within the defined range.

Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation in the numerical quantity that can occur, for example, through typical measuring and handling procedures in the real world; through inadvertent error in these procedures; through differences in the manufacture, source, or purity of the ingredients used to make the compositions or carry out the methods; and the like. The term “about” also encompasses amounts that differ due to different equilibrium conditions for a composition resulting from a particular initial mixture. Whether or not modified by the term “about”, the claims include equivalents to the quantities refers to variation in the numerical quantity that can occur.

As used herein the term “mushroom” or “filamentous fungi” shall be interpreted to include all tissues, cells, organs of the same, including but not limited to mycelium, spores, gills, fruiting body, stipe, pileus, lamellae, basidiospores, basidia, and the like.

As used herein the term “naturally-enhanced” with respect to whole foods such as mushrooms, yeast, cyanobacteria, Spirulina and Vitamin D, shall include pulsed UV irradiated mushrooms, yeast, cyanobacteria, Spirulina, etc. produced by the methods disclosed herein. The naturally-enhanced products according to the invention may include the enhanced whole food as well as powders and other forms obtained from the whole food. Still further the naturally-enhanced products of the invention may further include the product as well as any added minerals or other substances to a growing medium with resultant increase in contained ergothioneine or organic selenium.

The terms “subject” or “patient” are used herein interchangeably and as used herein mean any mammal including but not limited to human beings including a human patient or subject to which the compositions of the invention can be administered. The term “mammals” include human patients and non-human primates, as well as experimental animals such as rabbits, rats, and mice, and other animals.

The term “treating” or “treatment” as used herein, refers to any indicia of success in the prevention or amelioration of an injury, pathology or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a subject's physical or mental well-being. The treatment or amelioration of symptoms can be based on objective or subjective parameters; including the results of a physical examination, neurological examination, and/or psychiatric evaluations. Accordingly, the term “treating” or “treatment” includes the administration of the compounds or agents of the present invention which may be in combination with other compounds.

The term “weight percent,” “wt-%,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt-%,” etc.

Methods of Use

Embodiments of the invention include methods of preventing hair loss and/or nail deterioration, methods of treating the same, and/or methods for restoring or improving hair, nail and skin turgor and growth using Ergothioneine and/or Vitamin D. The methods of use disclosed herein may be employed for treating all types of hair loss and/or decreased hair and/or nail growth resulting from a variety of conditions, including skin disorders and/or diseases, such as psoriasis, alopecia greata and the like with Ergothioneine and/or Vitamin D, without limiting the scope of the invention.

Methods of the invention may also include the formulation and administration of an Ergothioneine serum for treating alopecia greata, psoriasis and/or other forms of hair, nail and/or skin loss and/or other related damage in a patient. The serum may be provided in the form of an extracted Ergothioneine serum, which is combined with another source of Ergothioneine, Vitamin D, chitin glucans, stem cells, other beneficial ingredients and/or combinations of the same. Use of serums according to the invention may include application to skin and hair, for example keratin treatments similar to those currently employed, and also for eyelash growth (such as Latisse®). The use of serums according to the invention demonstrate further cosmetic uses of the compositions according to the invention, in addition to the food and/or beverage compositions, pharmaceutical compositions, and the like disclosed herein.

Methods of the invention may be further used to control free radical and/or inflammation to protect skin, hair and nail health in humans using Ergothioneine and/or Vitamin D. Still further, methods of administering Ergothioneine and/or Vitamin D provide the donation of electrons required to maintain the body's zeta electric potential for normal skin healing, repair and/or aiding in adherence of grafted skin used in surgical repair. The methods include the administering, topical and/or orally of a source of Ergothioneine and Vitamin D to allow the Ergothioneine to act as a potent electron donor to maintain the skin's zeta electric potential.

Still further the methods of the invention use Ergothioneine and/or Vitamin D to act as an antioxidant and scavenger of free radicals to protect against oxidative skin damage. According to a particular embodiment of the invention, Ergothioneine neutralizes free radicals (e.g. through electron donation) in various tissues, including for example skin, hair and nail tissue providing beneficial results as disclosed herein. According to a further embodiment of the invention, the use of Ergothioneine and/or Vitamin D effects telomere length, namely the maintaining of telomere length in order to provide methods of preventing tissue from aging and/or other benefits. According to an embodiment of the invention, a method of treatment comprises, consists of and/or consists essentially of administering to an animal or patient in need thereof a source of Ergothioneine and optionally a source of Vitamin D, wherein upon administration of the same improves the treatment for skin, hair and/or nail growth, and/or treats a variety of skin, hair and/or nail conditions and/or disorders.

According to an aspect of the invention, the use of Ergothioneine and/or Vitamin D is efficacious in targeting genes in DNA. In an aspect, the use of Ergothioneine and/or Vitamin D for use in hair, skin and/or nail treatments provides a mechanism for reprogramming the DNA of such hair, skin and/or nail cells. In an aspect, the effect is to cause improvements in hair, skin and/or nail cells, such as for example looking younger and/or healthier based on such cellular reprogramming from the various methods of providing or administering Ergothioneine and/or Vitamin D according to the present invention. Beneficially, the use of Ergothioneine and/or Vitamin D according to the invention provide various compositions and methods for directly targeting genes and DNA in need of correction. For example, genes such as sirtuins (e.g. the youth gene) have been shown to affect the rate at which cells “age.” It would be beneficial according to the methods of the invention to reprogram and/or target genes, such as sirtuins, in hair, nail and/or skin.

According to an embodiment of the invention, the sources of Ergothioneine and Vitamin D may be naturally extracted and/or enhanced or synthesized. In an embodiment of the invention an enhanced source of Vitamin D may be obtained from a filamentous fungi, tissue, substrate, spent substrate or component thereof, with increased levels of Vitamin D. A suitable example is the novel mushroom whole food Ergo-D2™.

According to a further embodiment of the invention, a method of treatment comprises, consists of and/or consists essentially of increasing resistance to oxidative stress and/or free radicals through administering an effective amount of Ergothioneine, and optionally a naturally extracted and/or enhanced source of Vitamin D, such as a filamentous fungi that has been naturally enriched in Vitamin D2.

A still further embodiment of the invention includes a method of treating a disease state associated with inflammation and/or oxidative stress, including for example increased production of free radicals, comprising administering a composition comprising Ergothioneine and a pulsed UV irradiated, filamentous fungi, non-filamentous fungi, tissue, substrate, spent substrate or component thereof, with increased levels of Vitamin D2, wherein upon administration of the same, survivability is increased when compared to an animal with such disease state without such treatment. According to each of the embodiments of the invention the Ergothioneine may be obtained from the whole food sources and/or algae, such as cyanobacteria and Spirulina, as disclosed in this specification.

According to a further embodiment of the invention, a method of treatment comprises, consists of and/or consists essentially of reducing the levels of reactive oxygen species, free radicals, damaged DNA and/or toxic proteins in stem cells under oxidative stress through administering an effective amount of Ergothioneine, and optionally a naturally extracted and/or enhanced source of Vitamin D, such as a filamentous or non-filamentous fungi that has been naturally enriched in Vitamin D2. Such methods also promote DNA and protein repair within such stem cells allowing increased cell proliferation, differentiation and viability. Although not wishing to be limited to a particular theory of the invention, such increased cell viability results in increased hair, nail and skin growth, protection, and maintenance of normal hair and skin colors.

According to various embodiments of the invention, stem cells for skin, including without limitation hair and nail tissue, are provided Ergothioneine as a result of the ergothioneine transporter within the cells. In particular, the ergothioneine transporter within stem cells are provided Ergothioneine for the promotion of growth of the skin cells. The stem cells further are stimulated, such that stem cells are produced and/or the viability of the stem cells are improved through the administration of Ergothioneine. Still further, the ergothioneine transporter is activated through the providing of Ergothioneine to the stem cells.

Without intending to be limited according to a particular theory of the invention, a stem cell or other targeted tissue for the skin use the ergothioneine transporter (ETT) for uptake and delivery of Ergothioneine according to the invention. As ETT is a specific organic cation transporter (originally called OCTN1 and tetraethylammonium (TEA) was proposed as the substrate), and Ergothioneine is the best known substrate of ETT. The ETT is a protein integrated into the cell membrane. Without limiting the theory of the invention, as ETT is a member of the SLC22 family of transport proteins, it is expected both from hydropathy analysis and by analogy to be distantly related to bacterial transport proteins for whom crystal structures have been resolved to contain a core of 12 alpha-helical transmembrane segments (Grundemann, Prev. Med., 2012 May; 54 Suppl:S71-4). As the ETT controls and indicates ergothioneine activity according to the invention the ETT is responsible for transferring water-soluble substances through the lipophilic biological membranes of cells to exert pharmacological action or to facilitate elimination. These transporters are located in the outer cell membranes of the eukaryotic cell as well as mitochondria (a tubular shaped organelle found within the cytoplasm of cells) which supply cellular energy and are also involved in cell signaling, cellular differentiation, cell death as well as cell growth.

In one aspect, the ergothioneine transporter delivers Ergothioneine to a hair bulb, including the stem cells located within a hair bulb (as shown in FIGS. 1-2) for the growth of hair. In one aspect, the ergothioneine transporter delivers Ergothioneine to the stratum spinosum of the skin epidermis (as shown in FIG. 2) differentiation and proliferation of keratinocytes which form a barrier against environmental damage, such as pathogens, heat, UV radiation and water loss. In other aspects, the ergothioneine transporter in other stem cells for the skin delivers Ergothioneine to the targeted tissue for growth, such as nail growth as well as production and maintenance of natural hair, nail, and skin colors.

According to a preferred embodiment, ETT is identified in the cell membrane of the stem cell in the hair bulb. According to a further preferred embodiment, ETT is identified in the stratum spinosum of the skin epidermis. Without intending to be limited according to a particular theory of the invention, the stem cell in the hair bulb and/or in the stratum spinosum of the skin epidermis uses the ETT for uptake and delivery of Ergothioneine according to the invention, providing a basis for skin repair and hair growth in alopecia greata and other causes of hair loss, such as for example, hormonal changes, stress, etc. and/or other skin conditions benefiting from the delivery of Ergothioneine via the ETT.

The methods of the invention for delivery of Ergothioneine and/or Vitamin D are preferable provided through a food and/or beverage composition. Alternatively, the delivery of Ergothioneine and/or Vitamin D may be through nutraceutical compositions. Still further, the delivery of Ergothioneine and/or Vitamin D may be through pharmaceutical compositions. Exemplary embodiments of the compositions are disclosed in further detail herein. Preferably, the delivery of Ergothioneine and/or Vitamin D is directed to the targeted tissue, such as the hair bulb or stratum spinosum of the skin epidermis, through the blood supply such as through the consumption of a food, beverage, tablet, capsule or the like.

According to an embodiment, the delivery to the targeted tissue, such as the hair bulb and/or the stratum spinosum of the skin epidermis, is not only through local administration (i.e. topical application to the skin). According to a further embodiment, the delivery to the targeted tissue, such as the hair bulb and/or the stratum spinosum of the skin epidermis, is achieved through the blood supply and supplemented through a complementary topical delivery source (e g skin product delivered through topical absorption).

Methods of the invention may further include the delivery of Ergothioneine using noninvasive methods for transdermal delivery. These methods include chemical mediation using liposomes and chemical enhancers or physical mechanisms such as microneedles, iontophoresis, electroporation, and ultrasound (sonophoresis). Ergothioneine's coefficients of water solubility make this amino acid available for ultrasound mediated transdermal delivery. One hypothesis indicates that once the compound has traversed the stratum corneum, the next layer is easier to cross and subsequently the compound can reach the capillary vessels to be absorbed (Mitragotri et al., Science, (1995) 269:850-3).

Methods of the invention may further include the delivery of Ergothioneine as a result of its natural ability to follow water molecules between cells down to the hair bulb and/or stratum spinosum of the epidermis as a preferred form of local application. This is a result of the water solubility of Ergothioneine. According to an embodiment, the Ergothioneine may be delivered through the encapsulation and use of liposomes to enhance delivery efficiency. As one skilled in the art will ascertain, liposomes may be used in transdermal drug delivery systems because of its much higher diffusivity in skin compared to most compounds alone.

Methods of use according to the invention may include administration of the compositions, food products, supplements and/or pharmaceutical compositions on a daily basis, weekly basis, or other frequency for the particular purpose. Although not intending to be limited to a particular theory of the invention, it is believed that daily administration of the Ergothioneine and/or Vitamin D sources benefit a variety of disease states associated with inflammation and oxidative stress, including those resulting in or causing decreased skin, hair and/or nail growth, associated diseases and/or conditions, such as for example, psoriasis, alopecia greata or other hair loss and/or hair color conditions, or other comorbid hair and nail conditions.

In an aspect of the invention, daily supplementation is preferred for those with significant risk for such decreased skin, hair and/or nail growth or associated comorbid diseases, so that they are preloaded with the bionutrients and have elevated serum levels in order to protect against acute and chronic effects of the conditions. Supplementation on a regular and/or daily basis can also build up storage levels of the key bionutrients which can be mobilized at a time of physiologic need. According to this embodiment, daily supplementation reduces the signs and symptoms of hair loss and/or decreased nail growth, premature graying of hair and/or other skin diseases and/or conditions, such as for example psoriasis.

The methods according to the invention may further include the step of assessing, measuring and/or confirming the presence of the ETT in the target tissue to be treated. For example, the assessment, measurement and/or confirmation of the presences of the ETT within the skin, hair and/or nails may take place prior to treatment according to the invention. The measurement of ETT within the target tissue may include the identification or assessment of expression of ETT, including overexpression or under expression of ETT. In a further aspect, the measurement and/or assessment may be done in pathologic or histologic tissues, including for example tissues accumulated within tissue banks for various disease states, and conclusions drawn for application to the specific patient to be treated. In a further aspect, the measurement and/or assessment may comprise, consist of and/or consist essentially of confirming the existence of ETT within the skin, hair and/or nail tissue to be treated based upon prior documentation of such ETT presence from pathologic or histologic tissues.

In a further aspect, the step of assessment, measurement and/or confirmation of the presence of ETT takes place within a stem cell. As one skilled in the art will ascertain, stem cells are the building blocks of multi-cellular living organisms providing cellular signals directing undifferentiated cells to develop into a particular type of cell. According to an embodiment of the invention, the providing of Ergothioneine to a stem cell within the skin can be used to replace or repair damaged cells, and/or treat damaged cells responsible for the lack of hair and/or nail growth or other skin-related conditions and diseases. Ergothioneine has the ability to neutralize oxidative stress and free radicals within the mitochondrion of stem cells allowing adult stem cells to divide and renew indefinitely thereby generating new hair, nail and skin cells.

According to an embodiment of the invention the ETT is located within the bulb, also referred to as a hair follicle bulb. Recent evidence suggests the epithelial stem cell is located within the hair follicle bulb, which is part of the outer root sheath that is in continuity with the interfollicular epidermis and sebaceous gland. The use of ETT to deliver Ergothioneine promotes hair growth from the stem cells located at the base of the hair follicle and/or the stratum spinosum of the skin epidermis, whose function is protection of the skin from pathogenic invasion, water loss and UV damage. Additionally ETT may also be involved in melanocytes and melanin producing cells and be actively promoting hair color according to methods of use disclosed herein.

A further embodiment of the invention is to provide Ergothioneine to stem cells that are extracted from adult hair bulb or nail bulb tissue and are placed in a controlled culture that allows them to divide and replicate. A still further embodiment of the invention is to provide Ergothioneine to stem cells that are extracted from stratum spinosum of the skin epidermis and are placed in a controlled culture that allows them to divide and replicate. This stem cell line can be used to treat mammals, including humans, with hair loss, nail loss, and various skin conditions and/or diseases.

Compositions

According to an embodiment of the invention, a nutritional supplement, ingredient, food or beverage composition and/or pharmaceutical composition for improving skin, hair and nail growth may include Ergothioneine, Vitamin D2 and/or D3, phytonutrients, beta glucans, n-acetyl cysteine (NAC), turmeric and/or curcumin, omega-3 or alternative antioxidants, a pharmaceutically-acceptable carrier and/or combinations of the same. The various compositions according to the invention can be provided in ingestible formulations and/or topically (or otherwise as disclosed herein) applied. The various compositions may be formulated from naturally-available components and/or synthetically produced components, as set forth herein.

As used herein the term Ergothioneine shall be interpreted to include variants, homologs, optical isomers and the like which retain the antioxidant activity of Ergothioneine or L-Ergothioneine as demonstrated and described herein. Ergothioneine is a naturally-occurring amino acid. Ergothioneine is a unique, stable, natural antioxidant that cannot be made in human cells, but must be acquired exclusively from food. Ergothioneine from any suitable source may be used according to the invention. L-Ergothioneine is available commercially or from dietary sources such as mushrooms and the various sources disclosed herein according to the invention. The compound is also available from Actinobacteria, filamentous fungi, cyanobacteria, Spirulina, oats, barley and other whole food sources. Ergothioneine for use in compositions according to the invention may be obtained from an independent bionutrient source, such as Vitamin D enriched mushrooms disclosed herein, whole food sources, cyanobacteria and Spirulina as disclosed according to the embodiments of the invention.

According to one embodiment of the invention, Vitamin D2 and/or D3 may be provided from a UV irradiated, Agaricus fungi, tissue, substrate or component thereof with higher levels of Vitamin D2 or Vitamin D3 than a non-irradiated product. In another aspect, the Vitamin D2 and/or D3 may be provided from UV irradiation of 7-dehydrocholesterol extracted from lanolin, such as that found in sheep's wool. According to a preferred embodiment of the invention, the novel mushroom whole food (Ergo-D2™) may be used. Ergo-D2™ contains high levels of bioactive components previously shown to have health promoting properties and not produced by mammals—Vitamin D2, L-Ergothioneine (ET), organic selenium, beta-glucans and chitin-glucans. Without being limited to the content of the compositions employed in the methods of use of the invention, suitable compositions such as the Ergo-D2™ product have been demonstrated to contain Vitamin D2 as the most abundant product, and further including previtamin D2, lumisterol2 and tachysterol2, in order of decreasing abundance. Preferred Vitamin D and Ergothioneine enriched mushrooms according to the invention are pulsed with UV light at lower ranges and for very brief periods have increases by as much as 800 times the % DV (percent daily value) of Vitamin D content, per serving with no deleterious effects on the morphology or appearance of the mushroom. Pulsed UV-light treatments to increase Vitamin D₂ content in mushrooms were conducted with a laboratory scale, pulsed light sterilization system (SteriPulse®-XL 3000, Xenon Corporation, Woburn, Mass.) that is present in the Department of Agricultural Biological Engineering at Penn State University. Although it has been postulated that the UVB component of the Xenon pulsed light system is responsible for the enhanced Vitamin D and Ergothioneine effects, such exemplary systems use pulsed light including the entire spectrum of light and may also include other components that contribute to the beneficial effects, which are intended to be within the scope of the invention. Additional description of the suitable methods of enriching such mushrooms is disclosed for example in U.S. application Ser. Nos. 11/686,033, 12/502,677, 12/386,810, 12/887,276, 13/563,065, 13,644,867 and 13/684,662, which are herein incorporated by reference in their entirety.

Any type of mushroom, mushroom part, component, fungi or even used substrate for cultivating mushrooms, with ergosterol present may be used. This includes all filamentous and non-filamentous fungi where ergosterol has been shown to be present and includes the use of tissues such as the mycelia, spores or vegetative cells. Fungi as referred to herein also refer to the eukaryotic yeast and yeast products. This includes, but is not limited to, for example, Coprinus, Agrocybe, Hypholoma, Hypsizygus, Pholiota, Pleurotus, Stropharia, Ganoderma, Grifola, Trametes, Hericium, Tramella, Psilocybe, Agaricus, Phytophthora achlya, Flammulina, Melanoleuca, Agrocybe, Morchella, Mastigomycotina, Auricularia, Gymnopilus, Mycena, Boletus, Gyromitra, Pholiota, Calvatia, Kuegneromyces, Phylacteria, Cantharellus, Lactarius, Pleurotus, Clitocybe, Lentinula (Lentinus), Stropharia, Coprinus, Lepiota, Tuber, Tremella, Drosophia, Leucocoprinus, Tricholoma, Dryphila, Marasmius, and Volvariella.

In addition, the solid substrate can be any part of the mushroom or mold, including the mycelia, spores etc., so long as ergosterol is present in at least part of the tissue or cells. In yet another embodiment, the spent mushroom substrate upon which mushrooms are cultivated, was enriched in Vitamin D using pulsed UV light according to the invention. As one skilled in the art shall ascertain, mushrooms are usually produced by first preparing a substrate, such as corn, oats, rice, millet or rye or various combinations, prepared by soaking the grain in water and sterilizing the substrate before inoculation with mushroom spores or mushroom mycelia. Mycelia are the filamentous hyphae of a mushroom that collect water and nutrients to enable mushrooms to grow. The inoculated substrate is then held to promote colonization of the mycelia, at which point the mycelia-laced grains become “spawn”. This is usually done in individual spawn bags. The substrate provides the nutrients necessary for mycelium growth. The mycelium-impregnated substrate then develops under controlled temperature and moisture conditions, until the hyphae of the mycelium have colonized the substrate. The mycelium enriched product usually is harvested after about four to eight weeks from the beginning of the process, with the contents of the spawn bag possibly processed into dry powdered product. According to the invention, this spent substrate may also be enriched in Vitamin D upon application of pulsed UV irradiation.

Non-limiting examples of other fungal genera, including fermentable fungi, include: Alternaria, Endothia, Neurospora, Aspergillus, Fusarium, Penicillium, Blakeslea, Monascus, Rhizopus, Cephalosporium, Mucor, and Trichoderma.

Yeast are also included in the fungal genera, as non-filamentous fungi. There are over 1,500 species of yeast, which are heterotrophic organisms that may be either unicellular or multicellular as well as either aerobic or facultative anaerobes. Yeasts are eukaryotic microorganisms. Any type of yeast, yeast part, component, or even substrate for cultivating yeast, with ergosterol present may be used according to the methods and compositions of the invention.

A further suitable embodiment of the invention is the biosynthesis of ergothioneine. The use of synthesized ergothioneine is further embodied according to the invention. For example, ergothioneine can be synthesized through the use of submerged fermentation of mushrooms. According to a further embodiment, the fermented mushrooms can then be dried and exposed to UV light to increase Vitamin D2. According to a still further embodiment, the ergothioneine can further be extracted from fermented mushrooms and/or natural unfermented mushrooms and added into and/or combined with other compositions according to the invention, such as Ergo-D2.

In addition to the irradiated mushrooms according to an embodiment of the invention for providing a composition with enhanced Ergothioneine and Vitamin D, additional substrates for Ergothioneine may be irradiated to enhance the Ergothioneine content, including for example cyanobacteria and Spirulina. According to a further embodiment of the invention, cyanobacteria and/or Spirulina may be added as an additive ingredient to the irradiated mushrooms. According to a further embodiment of the invention, cyanobacteria and/or Spirulina may be irradiated and added to irradiated mushrooms.

Additional antioxidants may be beneficial in the compositions according to the invention. For example, turmeric and/or curcumin are phytonutrients that act as an antioxidant. An example of a suitable phytonutrient according to the invention is turmeric. Tumeric is available in various forms contains up to 5% essential oils and up to 5% curcumin, a polyphenol. Curcumin is the active substance of turmeric and curcumin is known as C.I. 75300, or Natural Yellow 3. The systematic chemical name is (1E,6E)-1,7-bis(4-hydroxy-3-methoxyphenyl)-1,6-heptadiene-3,5-Dione and exists in tautomeric forms—keto and enol.

According to an embodiment, the compositions of the invention comprise one or more of the following: phytonutrients, antioxidants (such as omega-3), beta glucans, and/or n-acetyl cysteine (NAC) in addition to the Ergothioneine and Vitamin D sources to provide improved and/or synergistic responses for the various methods of use disclosed herein.

Food or Beverage Compositions

In an aspect of the invention, the ingestible food or beverage compositions may be administered to a patient in need thereof, providing a nutritional approach to the therapeutic strategies disclosed herein according to the invention, including for example, returning the cellular levels of Ergothioneine to their homeostatic states. In additional aspects of the invention, the compositions provide an increased amount of Ergothioneine, Vitamin D and/or other antioxidants, phytonutrients and/or other beneficial compounds to provide further benefits for the patient.

In an aspect of the invention, a medical food or beverage is provided. As referred to herein, a medical food or beverage refers to a product that contains “natural” ingredients that have been enhanced or concentrated for medical use (value added). Such compounds are administered for a specific disease or condition. In an aspect, the compounds may be used as a stand-alone treatment and/or regimen, or in the alternative the compounds may be a complement to other pharmacologic therapies.

An embodiment of the present invention also provides medical foods and/or beverages comprising combinations of Ergothioneine and/or Vitamin D2, such as that which may be obtained from enriched mushrooms, including extracts, fractions thereof or compounds thereof or any combination thereof, phytonutrients and/or antioxidants. According to an embodiment, the medical foods and/or beverages of the invention comprise one or more of the following: phytonutrients, antioxidants (such as omega-3), beta glucans, and/or n-acetyl cysteine (NAC) in addition to the Ergothioneine and Vitamin D sources. The food compositions according to the invention may comprise enriched mushrooms from a variety of fungi sources as disclosed according to embodiments herein this description. Alternatively, the food compositions according to the invention may comprise Ergothioneine obtained directly from whole food sources, Spirulina or cyanobacteria.

The medical food is compounded for the amelioration of a disease, disorder or condition associated with or caused by oxidative stress and/or decreased levels of Ergothioneine. According to a preferred embodiment of the invention, food compositions are intended for human consumption for daily supplementation. Ranges of the amounts of each component of the food compositions can be adjusted as necessary for the supplementation of individual patients and according to the specific condition treated. Any variations in the amount of the ingredients may be utilized according to the desired composition formulation.

The food composition according to the invention may be prepared by any of the well-known techniques known by those skilled in the art, consisting essentially of admixing the components, optionally including one or more accessory ingredients. In one embodiment, the extracts, fractions, and compounds of this invention may be administered in conjunction with other additives and fillers known to those of skill in the art. Other compatible actives may be included in the food compositions of the present invention.

According to one embodiment of the invention, a beverage composition is provided. For particularly suitable applications, a beverage composition is provided on a daily basis. According to a further embodiment, a food supplement is provided on a daily basis, to ensure that the supplementation provides a whole food source of the Ergothioneine and Vitamin D. Although not intended to be limited according to a particular theory of the present invention, providing a whole food source administers various co-enzyme factors from the whole food providing additional supplementation and treatment benefits.

According to an alternative embodiment, an extracted source of the Ergothioneine and

Vitamin D (e.g. dried mushroom powder or Spirulina) can be added to the food or beverage composition. Beverage compositions may be provided in the form of a powder, cachet, droplets or other composition formulation for dilution within water or other aqueous liquid composition. For example, a dose of the beverage composition may be administered to a patient in need thereof, by adding a powder into a bottle of water. Additional formulation delivery mechanisms are included within the scope of the invention as one skilled in the art would modify or alter based on the description of the invention provided herein.

Pharmaceutical and/or Cosmetic Compositions

In an aspect of the invention, the pharmaceutical and/or cosmetic compositions may be administered to a patient in need thereof, providing a pharmaceutical and/or cosmetic approach to the therapeutic strategies disclosed herein according to the invention, including for example, returning the cellular levels of Ergothioneine to their homeostatic states. In additional aspects of the invention, the compositions provide an increased amount of Ergothioneine, Vitamin D and/or other antioxidants, phytonutrients and/or other beneficial compounds to provide further benefits for the patient.

In an embodiment of the invention, a pharmaceutical and/or cosmetic composition for treating a disease state and/or condition associated with oxidative stress and/or decreased levels of Ergothioneine comprises a combination of the following ingredients (in a variety of combinations, such that not every component is required according to various embodiments of the invention), a source of Ergothioneine, a source of Vitamin D2 (such as that which may be provided from a UV irradiated, enriched mushroom, tissue, substrate or component thereof with higher levels of Vitamin D2 than a non-irradiated product), and a pharmaceutically-acceptable carrier. The pharmaceutical and/or cosmetic compositions according to the invention may further comprise antioxidants, phytonutrients, beta glucans, n-acetyl cysteine (NAC) and/or other beneficial components for treatment of the conditions disclosed herein.

According to a further embodiment of the invention, the pharmaceutical and/or cosmetic composition may further comprise another bioactive nutrient attached to Ergothioneine. Although not intended to be limited to a particular theory of the invention, the attachment of a bionutrient to Ergothioneine delivers the bionutrient along with the Ergothioneine, wherein the Ergothioneine acts as an active carrier to deliver the bionutrient to a cell or mitochondria within the cell. According to an additional non-limiting theory of the invention, the ETT permits the bionutrient to enter the cell or mitochondrial membrane. For example, selenium and/or extracted products from beer hops, oats, barley, etc. can be added to the Ergothioneine and the pharmaceutical compositions of the invention.

The pharmaceutically-acceptable carrier according to the invention facilitates administration of the composition to a patient in need thereof. Ergothioneine and compounds such as turmeric, N-acetyl cysteine, etc., extracts, fractions and/or compounds may be mixed with any of a variety of pharmaceutically-acceptable carriers in order to allow for a particular mode of administration and/or delivery.

“Pharmaceutically acceptable” as used herein means that the extract, fraction thereof, or compound thereof or composition is suitable for administration to a subject to achieve the treatments described herein, without unduly deleterious side effects in light of the severity of the disease and necessity of the treatment. According to the invention, the carrier may be a solid or a liquid, or both, and is preferably formulated with the compound as a unit-dose formulation, for example, a tablet, which may contain from 0.5% to 95% by weight of the active compound.

The pharmaceutical and/or cosmetic composition according to the invention may be prepared by any of the well-known techniques of pharmacy consisting essentially of admixing the components, optionally including one or more accessory ingredients. In one embodiment, the extracts, fractions, and compounds of this invention may be administered in conjunction with other medicaments known to those of skill in the art. Other compatible pharmaceutical and/or cosmetic additives and actives may be included in the pharmaceutically acceptable carrier for use in the compositions of the present invention.

Dose ranges of the pharmaceutical and/or cosmetic compositions can be adjusted as necessary for the treatment of individual patients and according to the specific condition treated. Any of a number of suitable pharmaceutical and/or cosmetic formulations may be utilized as a vehicle for the administration of the compositions of the present invention and maybe a variety of administration routes are available. The particular mode selected will depend of course, upon the particular formulation selected, the severity of the disease, disorder, or condition being treated and the dosage required for therapeutic efficacy.

The methods of this invention, generally speaking, may be practiced using any mode of administration that is medically acceptable, meaning any mode that produces effective levels of the active compounds without causing clinically unacceptable adverse effects. Such modes of administration include ingestible (i.e. oral), rectal, topical, nasal, transdermal or parenteral routes and the like. Accordingly, the formulations of the invention include those suitable for oral, rectal, topical, buccal, sublingual, parenteral (e.g., subcutaneous, intramuscular, intradermal, inhalational or intravenous) and transdermal administration, although the most suitable route in any given case will depend on the nature and severity of the condition being treated and on the nature of the particular active product used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above).

In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations of the present invention suitable for parenteral administration conveniently comprise sterile aqueous preparations of the active compound, which preparations are preferably isotonic with the blood of the intended recipient. These preparations may be administered by means of subcutaneous, intravenous, intramuscular, inhalational or intradermal injection. Such preparations may conveniently be prepared by admixing the compound with water or a glycine buffer and rendering the resulting solution sterile and isotonic with the blood. Alternately, the extracts, fractions thereof or compounds thereof can be added to a parenteral lipid solution.

Formulations of the inventive mixtures are particularly suitable for topical application to the skin and preferably take the form of an ointment, cream, lotion, paste, gel, shampoo, soap, spray, aerosol, or oil. Carriers which may be used include Vaseline, lanoline, polyethylene glycols, alcohols, transdermal enhancers, and combinations of two or more thereof.

Formulations suitable for transdermal administration may also be presented as medicated bandages or discrete patches adapted to remain in intimate contact with the epidermis of the recipient for a prolonged period of time. Formulations suitable for transdermal administration may also be delivered by iontophoresis (passage of a small electric current to “inject” electrically charged ions into the skin) through the skin or through ultrasound sonophoresis. For this, the dosage form typically takes the form of an optionally buffered aqueous solution of the active compound. Suitable formulations comprise citrate or bis/tris buffer (pH 6) or ethanol/water and contain from 0.01 to 0.2M active ingredient.

The therapeutically effective dosage of any specific compound will vary somewhat from compound to compound, patient to patient, and will depend upon the condition of the patient and the route of delivery. As a general proposition, a dosage from about 0.01 to about 50 mg/kg will have therapeutic efficacy, with still higher dosages potentially being employed for oral and/or aerosol administration. Toxicity concerns at the higher level may restrict intravenous dosages to a lower level such as up to about 10 mg/kg, all weights being calculated based upon the weight or volume of the enriched mushrooms, fractions thereof or compounds thereof of the present invention, including the cases where a salt is employed.

Extraction of Ergothioneine from Various Sources for Use in Compositions of the Invention

The isolation, extraction and/or sourcing of Ergothioneine from additional sources is disclosed according to the methods of use of the present invention. As a result, various whole sources of food and/or bacteria may be used to provide the Ergothioneine required for the methods of use and/or the compositions according to the invention.

Ergothioneine can be sourced from a non-enriched or an enriched source, such as the mushroom sources disclosed herein. In an embodiment, mushrooms are grown on substrates containing histidine to produce higher amounts of Ergothioneine. Exemplary mushroom sources are those having naturally higher levels of Ergothioneine, such as pleurotus eryngii. In additional embodiments, the mushrooms may be further enriched with Vitamin D2 and/or D3 and could be obtained, for example, from a UV irradiated, Agaricus fungi or Pleurotus fungi (or others as disclosed herein), tissue, substrate or component thereof with higher levels of Vitamin D2 than a non-irradiated product. A preferred source for the enriched mushroom is the whole food (Ergo-D2™), containing high levels of three bioactive components—Vitamin D2, L-Ergothioneine (ET) and chitin-glucans.

As one skilled in the art will ascertain based upon the disclosure of the invention, various other mushroom species are suitable for use as sources for ergothioneine. According to an embodiment of the invention, any type of mushroom, mushroom part, component, fungi or even used substrate for cultivating mushrooms, with ergosterol present may be used. This includes all filamentous fungi where ergosterol has been shown to be present and includes the use of tissues such as the mycelia, spores or vegetative cells. This includes, but is not limited to, for example, Coprinus, Agrocybe, Hypholoma, Hypsizygus, Pholiota, Pleurotus, Stropharia, Ganoderma, Grifola, Trametes, Hericium, Tramella, Psilocybe, Agaricus, Phytophthora achlya, Flammulina, Melanoleuca, Agrocybe, Morchella, Mastigomycotina, Auricularia, Gymnopilus, Mycena, Boletus, Gyromitra, Pholiota, Calvatia, Kuegneromyces, Phylacteria, Cantharellus, Lactarius, Pleurotus, Clitocybe, Lentinula (Lentinus), Stropharia, Coprinus, Lepiota, Tuber, Tremella, Drosophia, Leucocoprinus, Tricholoma, Dryphila, Marasmius, Yeast, and Volvariella.

According to a further embodiment of the invention, Ergothioneine can further be obtained from cyanobacteria. Cyanobacteria can be used for extraction of Ergothioneine and/or a source for Ergothioneine. Spirulina is blue-green algae that have been identified to be a source of Ergothioneine. Spirulina is a microscopic blue-green algae in the shape of a spiral coil, living both in sea and fresh water. It is the most common name for human and animal food or nutritional supplement made primarily from two species of cyanobacteria: Arthrospira platensis and Arthrospira maxima.

According to a further embodiment, various plant materials are used to source Ergothioneine for the methods of use and/or the compositions according to the invention. Plant material sources for Ergothioneine may include cereal grains, including oats, wheat and barley. Ergothioneine may be further extracted from beer hops, and cereal grains, including oats, barley, etc.

Upon extraction or isolation of Ergothioneine from a source additional molecules and entities can be attached to permit delivery into the cell along with the Ergothioneine. As is recognized in the art relating to Ergothioneine, ETT provides a mechanism of delivery of Ergothioneine within cells. As a result, it is desirable to attach additional molecules to Ergothioneine, upon isolation from at least the sources disclosed herein (e.g. whole foods and cyanobacteria), including for example, beta-glucans, chitin-glucans, antioxidants, selenium, phytonutrients, and/or vitamins, such as Vitamin C and Vitamin D2. The attachment of additional molecules to an extracted source of Ergothioneine permits the effective delivery into the mitochondria of the cells of a patient in need of treatment according to the embodiments of the invention.

The various embodiments of the invention, including methods of use or administration of compositions for the various treatments and/or methods to improve skin, hair and/or nail growth or related conditions associated therewith, are useful for a variety of subjects. Mammals may be treated using the methods of the present invention and are typically human subjects.

All publications and patent applications in this specification are indicative of the level of ordinary skill in the art to which this invention pertains. All publications and patent applications are herein incorporated by reference to the same extent as if each individual publication or patent application was specifically and individually indicated by reference.

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### Example 1

Pulses of UV radiation of approximately 1-10 J/cm² per pulse, preferably 3-8 J/cm² and most preferably 5-6 J/cm² are used to UV-enhance Vitamin D and/or its derivatives in filamentous fungi. Voltages may also vary based upon safety concerns but should generally be in the range of 1 to 10 or even up to 100 or 10,000 volts as safety mandates. The pulses should generally be in a range of 1-50 pulses per second more preferably 1-30 pulses per second and most preferably 1-10 pulses per second for a range of treatment post-harvest of 0 to 60 seconds.

The inventors used 5.61 J/cm² per pulse on the strobe surface for an input voltage of 3800V and with 3 pulses per second. Sliced mushrooms (Agaricus bisporus, white strain) were placed in the pulsed UV-light sterilization chamber and treated with pulsed light for up to a 20-second treatment at a distance of 17 cm from the UV lamp or 11.2 cm from the window. Control samples did not undergo any pulsed UV treatment. Treated mushrooms were freeze-dried and then sent to a selected commercial laboratory for Vitamin D2 analysis. In this study, a pulsed UV system was also evaluated for effects on the appearance of fresh mushroom slices during a shelf life study.

Results of the experiments demonstrated that pulsed UV-light was very effective in rapidly converting ergosterol to Vitamin D2. Control mushrooms contained 2 ppm dry weight. Vitamin D2, while 10 and 20 seconds of exposure to pulsed UV-light resulted in 17 and 26 ppm Vitamin D2, respectively. This increase was equivalent to over 1800% DV Vitamin D in one serving of fresh mushrooms after a 20 second exposure to pulsed UV. The mushrooms treated for 20 seconds also showed no noticeable difference in appearance initially as well as after 10 days of storage at 3° C. compared to the untreated control.

These results compared favorably to the previous pilot study (Feeney, 2006) where mushrooms were exposed to 5 minutes of conventional UV-light exposure. In that study, the mushrooms contained 14 ppm Vitamin D2, but they were also significantly discolored. Hence, the pulsed UV method shows considerable promise as a rapid means to enhance Vitamin D2 levels in fresh mushrooms, theoretically reducing required exposure times from minutes to seconds. Pulsed UV-light exposure did not result in any negative effects on mushroom quality.

Another experiment revealed that pulsed UV-light could rapidly convert ergosterol present in dried oyster mushroom powder to Vitamin D2 (Table 1). These findings indicate that this technology could be used to enrich other mushroom products with Vitamin D2.

TABLE 1
Vitamin D2 generation in dried oyster mushroom
powder exposed to pulsed UV-light (C-type lamp).

| Time of Exposure(s) | Vitamin D2 (PPM) |
|---|---|
| 0 | 8.5 |
| 8 | 15.18 |
| 16 | 24.24 |

The filamentous fungi product is subjected to pulsed UV irradiation after harvest, being irradiated with UV light for a time sufficient to enhance the Vitamin D content thereof. By utilizing UV irradiation, the food product has a substantially increased level of Vitamin D. Preferably, the food product is irradiated with UV radiation, specifically Ultraviolet-B (UV-B), a section of the UV spectrum, with wavelengths between about 280 and 320 nm, or Ultraviolet-C (UV-C), with wavelengths between about 200 and 280 nm. In a more preferred embodiment the UV radiation is pulsed. It is believed that the additional Vitamin D is obtained through the conversion of ergosterol due to the UV irradiation. The time may be the same or increased when the irradiation occurs during the growing process, or post-harvest though the UV irradiation can occur during both periods.

### Example 2

The work of Gründemann et al. demonstrates additional sources of Ergothioneine (ET) biosynthesis, including species of cyanobacteria (synthesis confirmed by the detection of the intermediate hercynine). The highest ET content of cyanobacteria in the examined samples was close to 1 mg per g dry mass, which approaches the same level as the top values (1-2 mg per g dry mass) reported previously for several mushrooms. As a result, it is demonstrated that cyanobacteria are a “high density” source of ET.

Previously, the biosynthesis of ET has been demonstrated only in fungi (including edible mushrooms) and mycobacteria, but these are unlikely sources for fishes. In the present study, the origin of ET accumulated in zebra fish was examined. There was virtually no ET, measured by LC-MS, in most tank vegetation (plant, green and red alga). However, ET was detected in a Phormidium sample, a cyanobacterium. In commercial fish feed preparations, ET content increased with the content of cyanobacteria Arthrospira platensis or Arthrospira maxima (Spirulina). High levels of ET (up to 0.8 mg per g dry mass) were measured in cyanobacteria preparations sold as dietary supplements for humans and in fresh Scytonema and Oscillatoria cultures. Thus, cyanobacteria can contain as much as or even more ET than King Oyster mushrooms (Pleurotus eryngii) which we measured at 0.4 mg per g dry mass. All samples with substantial ET content also contained the biosynthesis intermediate hercynine; this strongly suggests that cyanobacteria synthesize ET de novo and can produce high levels of Ergothioneine. Spirulina is a novel, safe, accessible, and affordable source of Ergothioneine for humans.

### Example 3

A study was conducted to confirm the role for medical food compositions for use in ameliorating symptoms of Parkinson's Disease (PD). One of the hallmark signs of PD is an aggregation of the protein alpha-synuclein which is found not only in the mid-brain but also in the brain stem and the olfactory bulb. These areas of the brain correlate to non-motor functions such as sense of smell and sleep regulation which could explain the non-motor symptoms experienced by some people with PD before any motor signs of the disease appear. Alpha-synuclein has been identified as a genetic risk factor for PD and is a logical target for therapies such as the medical food Ergo-D2™, which has the potential to inhibit synthesis and accumulation as well as lower the toxicity of this protein.

Matched cohorts of SNCA transgenic mouse model of PD were used to measure the therapeutic efficacy of ErgoD2™ to control movement disorder, improve grip strength, and lower alpha-synuclein in the cortex and/or midbrain of the PD animals. Additionally, alpha-synuclein was measured in the plasma, and glutathione was measured in the plasma, cortex and midbrain. IL-6 measurements and an immunohistochemistry study will also be performed on intestinal tissues to see if there are pathologic variances in intestinal function between cohorts, which could be associated with disease onset and progression.

Results: ErgoD2™ enhanced grip strength in all treated subjects, with females exhibiting the greatest therapeutic response. Balance/coordination was also enhanced in treated females compared to both the untreated and wild type. These results are in agreement with several reports in the literature that female mice perform better than males in rotor rod testing. Post-hoc contrast testing revealed that the mice treated with ErgoD2™ performed better than those treated with placebo (p=0.05).

Table 2 shows measured alpha-synuclein levels, which are further illustrated in FIGS. 3A-B and shown as alpha-synuclein concentrations (μg/mL homogenized midbrain lysate).

TABLE 2
Alpha-synuclein Levels

| Cortex | Mid-Brain | Plasma |
|---|---|---|
| Statistically significant | ErgoD2 ™ decreased | Alpha-synuclein in the |
| changes were not | alpha-synuclein levels, | plasma increased |
| observed. | as shown in FIG. 3A. | significantly with |
| | | ErgoD2 ™ therapy, |
| | | as shown in FIG. 3B. |

Alpha-synuclein aggregation in the substantia nigra is toxic to dopaminergic neurons of the mid-brain and is a pathogenic hallmark of PD and related movement disorders. Defined point mutations and gene duplications of the alpha-synuclein gene can also cause familial onset PD with 100 percent genetic inheritability. Midbrain mean alpha-synuclein concentration (μg/mL homogenized midbrain lysate) in each treatment group of Parkinson's model mice were measured by ELISA protocol. Statistically significant differences were found between groups (ErgoD2™ at the p=0.001 level).

A correlation was observed between the rise in alpha-synuclein levels in the plasma of treated mice and improved rotor rod and grip test performances, which is consistent with three independent studies that showed decreased Alpha-synuclein levels in PD patients when compared to age matched non-affected individuals (Li et al., 2007; Shi et al., 2012a; Shi et al., 2012b). Alpha-synuclein is certainly a protective agent and appears to be able to promote protective antioxidant functions in situations of stress where there is an accumulation of the antioxidant. Alpha-synuclein is at its highest abundance in immature reticulocytes and thus appears to act as a promoter of erythropoiesis. The complete absence of alpha-synuclein in knockout mice certainly impairs mitochondrial electron transport. Thus, in stressed situations, alpha-synuclein may well be crucial for restorative mitochondrial production of heme (hemoglobin) in reticulocytes during erythropoiesis.

Table 3 shows measured glutathione levels, which are further illustrated in FIGS. 4A-C showing the measured glutathione concentration.

TABLE 3
Glutathione Levels

| Cortex | Mid-Brain | Plasma |
|---|---|---|
| There was a clear | Glutathione in the | Glutathione in the |
| trend towards | mid-brain increased | plasma increased |
| higher cortex | significantly with | significantly with |
| glutathione with | ErgoD2 ™, as | ErgoD2 ™, as |
| ErgoD2 ™, as | shown in FIG. 4B. | shown in FIG. 4C. |
| shown in FIG. 4A. | | |

Untreated transgenic control had significantly lower glutathione concentrations than the wild type mice (p=0.066). Concentrations of glutathione present in midbrain homogenates (nM), as measured by glutathione assay.

Concentrations of glutathione present in cortex homogenates (nM), as measured by glutathione assay. Error bars represent±standard error. Glutathione is a major protective antioxidant in animal cells. It is present most abundantly in its reduced form (GSH) in healthy cells. The levels of GSH and glutathione disulfide (GSSG), the oxidized form, are used as indicators of oxidative stress and overall cell health. One of the well-established markers of PD is a decrease in glutathione levels in the substantia nigra (SN). This decrease is specific to the SN and is not observed in other sections of the PD brain.

In agreement with this model, we found no significant differences in cortex glutathione concentration between knockout mice and either untreated or NAC-treated transgenic Parkinson's model mice, but we saw a decrease in glutathione levels in the corresponding transgenic mid-brains compared to the knock-out mouse mid-brains. In treated transgenic midbrains, however, glutathione concentrations were much higher than the untreated transgenic controls. There was no significant difference in glutathione levels between treated transgenic mid-brains and the knock-out mouse midbrains, suggesting that ErgoD2™ is having a positive effect on the oxidative stress in the transgenic mice and returning mid-brain glutathione to healthy concentrations. Interestingly, in cortex samples, the treated mice showed some evidence of having higher glutathione levels than any of the other treatment groups, including the knock-out mice.

Study Conclusions: Alpha-synuclein (AS) is a protein that, in humans, is encoded by the SNCA gene. AS accumulates in Parkinson's disease as fibrillar aggregates as hallmark features in affected brain regions, most notably in mid-brain nigral dopaminergic neurons. The levels of AS in neurons may be critical to the protein's toxicity and the focus of this study was to evaluate the ability of the medical food ErgoD2™ to regulate levels of AS.

The results show the potential of ErgoD2™ to not only lower AS in the midbrain of PD mice, but also to increase levels in the plasma. This increase in plasma AS levels was also accompanied by significant elevation of glutathione, an important antioxidant cellular protectant. AS may be playing a protective antioxidant role outside the brain and only when transformed into its fibrillar form does it become toxic. Support for this pathogenic mechanism is further shown by the ability of the potent antioxidant ErgoD2™ to improve motor function and grip strength in the diseased animals.

This study has additional correlations to the use of ergothioneine and Vitamin D2, such as the ErgoD2 medical food composition for providing a means to increase glutathione in a patient in need thereof. The results showing an increase in glutathione in the cortex, mid-brain and plasma are important as there is a strong correlation between the cellular role and importance of both ergothioneine and glutathione; wherein the role of ergothioneine may be as valuable as glutathione. The combination of the presence of Ergothioneine and/or its ability to either lower breakdown of glutathione or increase production in the face of oxidative stress is most important. Medical researchers have shown a positive correlation between glutathione content and the percentage of anagen hairs present in a scalp sample, concluding that glutathione helps maintain the hair growth cycle.

### Example 4

A clinical trial for evaluation of Ergothioneine and Vitamin D2 compositions for use as a Dietary Supplement and/or Medical Food to treat patients with metabolic syndrome and/or insulin resistance (including both Type I and Type II diabetes) was conducted. The trial evaluated symptom response, hemoglobin A1C and other biomarker changes in metabolic syndrome and/or insulin resistance. In addition, changes in hair and nail growth in the patient populations were evaluated. The trial was initiated in Bonaire, Netherlands Antilles beginning Aug. 1, 2012. The trial is an open clinical study with no placebo. The trial is double blinded and includes between 25-35 patients, between 30-70 years old. All study patients are compared against his/herself.

The trial methodology was as follows: Male and female patients with diagnosed Type I or Type 2 diabetes were enrolled. All subjects were evaluated at enrollment including but not limited to a standard physical examination and the following diabetes standard of care blood tests: insulin levels, glucose levels including hemoglobin A1C, complete blood count including mean corpuscular hemoglobin, c-reactive protein, cardiac profile including cholesterol, HDL, LDL, etc. Re-evaluation is performed at 30, 60, 90 and 120 days.

All subjects are instructed to take 2 capsules of the Medical Food, ErgoD2™ between 7 am and 10 am on days 0-3 and to increase to 4 capsules between 7 am and 10 am on day 4. Questionnaires were used to evaluate symptom response, including but not limited to joint pain, energy, sleep patterns, and hair and nail growth. The ErgoD2 Food Product Information ErgoD2™ 2,000 mg, Ergocalciferol (Vitamin D2) 11,000 IU's, L-Ergothioneine 3 mg.

The study results to date include an enrollment of 30 patients. After 60-90 days of evaluation and treatment 13 patients have responded to a circulated questionnaire and 7 patients have noted significant increase in hair and nail growth. Notably, this response indicates approximately a greater than 50% improvement in hair and nail growth from the responsive patient population. The confirmed presence of the ETT in tissues similar to those of the trial patient population is shown in FIG. 9, wherein pancreatic tissue cells of the Islet of Langerhans in a normal patient (non-diabetic) show faintly expressed ETT, in comparison to the strongly expressed ETT cells of a Type 1 diabetic. FIG. 9 shows a clear detection of the ETT within the pancreatic cell and provides support for the need for supplying this patient population with Ergothioneine.

This initial data is important as it demonstrates a physiologic connection between diabetes and/or its attendant metabolic disorders with hair and/or nail growth. Scientists have found, within a diabetic patient's immune system, an alternative macrophage that releases signaling molecules that lead to insulin resistance in certain bodily tissues, such as the liver. Metabolic issues including a metabolic endotoxemia, which may be a response to dietary fats and processed sugars, is associated with diabetes, stimulation of the alternative macrophage and resultant insulin resistance (metabolic syndrome) pathologies. This condition has been recognized by Odegaard & Chawla who showed the relationship between these diseases and conditions with the associated inflammatory response in metabolic tissues (Annu. Rev. Pathol. Mech. Dis., 6:275-97 (2011)). Moreover, similar corrupted immune responses, in the translation process from the gene, have been recognized in autoimmune conditions (including Type 1 diabetes) indicating that immune and tissue stem cell reprogramming could provide a treatment mechanism and/or cure for such conditions. Such research is important as it provides an association between macrophages and these conditions, including the initiation of the metabolic inflammatory response, which is consistent with the method of using the compositions of the invention for reducing free radicals and chronic inflammation in order to provide a protective role in skin, hair and nail health. In addition, this pathogenic association suggests a role for supplementing people with metabolic syndrome/diabetes with Ergothioneine and/or dietary Vitamin D as a treatment for male and female pattern baldness, Alopecia Areata, and other hair loss disease conditions.

### Example 5

Various commercial antibodies against the transporter protein were tested on normal human epididymis, placenta, skin with hair follicles, and bone marrow to determine their pattern of expression and enable further characterization and comparison of the antibodies to target tissues. The purpose of this study is to evaluate the expression pattern of SLC22A4 in a variety of human tissues and diseases through immunohistochemistry. Five commercial antibodies to SLC22A4 were evaluated on formalin-fixed, paraffin-embedded (FFPE) positive SLC22A4 and negative control cell lines and a multi-tissue array of human normal tissues to identify the best reagents and concentrations for use in this immunohistochemistry study. The cell lines tested were a positive cell line TNCS1a-ETTh, which expresses SLC22A4, and a negative control cell line TNCS1a-CTTh, which expresses a carnitine transporter.

Methods:

The tissues tested in this study were formalin-fixed, paraffin embedded samples of epididymis, placenta, scalp skin, bone marrow, kidney, intestine, and foreign body granuloma with associated immune response. IHC was performed using steam based antigen retrieval (pH 6.0 citrate buffer), and antibodies were used at varying concentrations: 2 ug/ml, 40 ug/ml, and at dilutions of 1:200.

Antibodies were titrated prior to treatment of the slides. The slides were then treated with the antibodies, followed by the application of an anti-rabbit secondary antibody (i.e. principal detection system), and an AP-Vector Red detection system for the production of a fuchsia-colored deposit. Serial sections were also evaluated in the absence of primary antibody to determine the background produced by the secondary antibody and detection system. The slides were interpreted by a pathologist and each antibody was evaluated for the presence of specific signal, level of background, and concordance with expression results reported in the literature. Staining was recorded on a 0-4 scale (0=negative, 1=blush, 2=faint, 3=moderate, 4=strong).

Results:

As shown in FIG. 5 the positive control was a cell line expressing SLC22A4. The negative control is further shown in FIG. 5 using a cell line expressing carnitine. The clarity and sensitivity of the assay is revealed by the single cell stained with vector red.

FIG. 6 shows the positive control in kidney tissue, obtained from normal kidney tissue from a 68-year old male. The proximal convoluted tubules (PCT) show moderate staining, validating the presence of ergothioneine and strongly suggesting that ergothioneine is absorbed back into the body by the PCT. This finding as well as previous studies on urine samples by scientists in the field (Dr. Gründemann) demonstrates that the body does not eliminate ergothioneine as a waste product through the urine produced by the kidneys. Instead, this important beneficial cytoprotectant compound is reabsorbed by the body.

FIG. 7 shows the heavy staining of macrophages in the foreign body granuloma. The tissue was obtained from the epididymis of a 61-year old male. FIG. 8 shows the moderate to strong staining of macrophages, and the faint to moderate staining of megakaryocytes in the normal bone marrow of a patient. The bone marrow was obtained from an autopsy of a 74-year old male. These findings reveal the significant overexpression of the Ergothioneine Transporter in bone marrow progenitor cells which differentiate into adult macrophage killer cells and blood thrombocytes (platelets) which are necessary for normal blood clotting. The megakaryocytes are derived from hematopoietic stem cell precursor cells in the bone marrow. These multipotent stem cells live in the marrow sinusoids and are capable of producing all types of blood cells depending on the signals that they receive. In addition to the bone marrow, they are primarily produced by the liver, kidney, and spleen.

### Example 6

Additional immunohistochemistry studies were conducted to evaluate the expression pattern of SLC22A4 in human tissue samples of patients having alopecia greata scalp skin compared to normal scalp skin. The antibody disclosed in Example 5 having a high specificity for SLC22A4 was evaluated at 2 μg/ml on four samples of normal scalp skin and two samples of scalp skin from patients with alopecia greata.

The principal detection system consisted of a Vector anti-rabbit secondary (BA-1000) and a Vector ABC-AP kit (AK-5000) with a Vector Red substrate kit (SK-5100), which was used to produce a fuchsia-colored deposit. Tissues were also stained with positive control antibodies (CD31/Vimentin cocktail) to ensure that tissue antigens were preserved and accessible for immunohistochemical analysis. Only tissues that were positive for CD31 and vimentin staining were selected for the remainder of the study. The negative control consisted of performing the entire immunohistochemistry procedure on adjacent sections in the absence of primary antibody. The slides were interpreted by a pathologist and each antibody was evaluated for the presence of specific signal and level of background. Staining was recorded on a 0-4 scale (0=negative, 1=blush, 2=faint, 3=moderate, 4=strong). Slides stained at 2.0 μg/ml were imaged with a DVC 1310C digital camera coupled to a Nikon microscope. Images were stored as TIFF files with Adobe Photoshop.

Samples of alopecia greata showed increased staining within the epidermis. Staining was occasionally patchy and irregular, with focal areas of increased staining Increased staining was also observed in melanocytes, hair follicles, reactive fibroblasts, and inflammatory cells. Whereas melanocytes within normal epidermis were only occasionally faint to moderate, both samples of alopecia showed increased staining within melanocytes. In addition, hair follicles in normal skin samples tended to show strongest staining at the bulb, with only blush to faint staining of the internal root sheath and mostly negative staining of the external root sheath along the length of the hair, but in the two alopecia samples, keratinocytes showed higher levels of staining along the entire length of the hair, including within the infundibulum, the isthmus, and the supra bulbar region.

Results:

Sample 1: A sample of scalp was obtained from a 17-year-old female who died of trauma. The epidermis showed faint to moderate staining, with diminished staining of epithelium at the infundibulum. Within the stratum spinosum, staining was slightly increased in the upper layer, with negative staining of the stratum granulosum and corneum, as shown in FIG. 10. Hair follicles showed blush staining of the inner root sheath, with strong staining of the matrix keratinocytes of the hair root and faint staining of papilla, as shown in FIG. 11. Melanocytes were occasionally faint. Sebaceous glands were negative and eccrine glands were faint. Adipocytes were mostly negative. Dermal vessels showed rare faint staining of endothelium and smooth muscle. Schwann cells within nerves were negative or showed blush staining. Macrophages were faint to moderate.

Sample 2: This sample of scalp was obtained at surgery from a 22-year-old female. The epidermis was moderately positive, with fairly uniform staining from the stratum basalis to through the stratum spinosum. The stratum granulosum and corneum were negative. Hair follicles showed faint staining throughout the infundibulum, with moderate to strong staining of the matrix keratinocytes of the hair root and faint staining of papilla. The hair shaft was negative, but the inner root sheath was faint to moderate, and the outer root sheath was negative to faintly positive. Melanocytes were faint. Sebaceous glands were faint and eccrine glands were faint to moderate. Adipocytes were mostly negative. Dermal vessels showed faint to moderate staining of endothelium and faint staining of smooth muscle. Schwann cells within nerves were also moderate. Macrophages were faint to moderate.

Sample 3: This sample of scalp was obtained from a 20-year-old male who died of injuries related to a motor-vehicle accident. The epidermis was faint with punctate areas of moderate staining in the stratum spinosum, as shown in FIG. 12. Hair follicles showed negative or blush staining within the outer root sheath of the infundibulum and shaft, with faint staining of the inner root sheath. Strong staining was observed at the bulb, within matrix keratinocytes and melanocytes. Melanocytes in the epidermis were occasionally faint. Sebaceous glands were negative and eccrine glands were negative to blush. Adipocytes were mostly negative. Within dermal vessels endothelium was negative.

As shown in FIG. 13 the Ergothioneine Transporters (ETT's) are located within the stem cells of a hair bulb. The antibodies are used to show the ETT within the hair bulb. FIG. 13 shows that antibodies have largely negative staining of the outer root sheath clear cells, with faint positive staining of the inner root sheath. The dark colored melanin granules within the melanogenic zone are further shown in FIG. 14. FIG. 14 shows the strong staining in the hair root and the shaft, above the papilla, including the basal reserve cells. Although there are insufficient follicles in the depicted immunohistochemistry sample to trace out the entire growth pattern, according to the invention the ETT is located within the stem cells of the hair, and various phases of hair growth, including hair color, are affected according to the methods of the invention. As the hair shaft grows and differentiates, the intensity of staining lessens, as compared to the intensity with the initial stem cells.

Sample 4: This sample of scalp was obtained from a 40-year-old male who died of injuries related to a motor vehicle accident. The epidermis showed faint to moderate staining, with stronger staining both along the bottom third within the dermal papillae, as well as close to the stratum granulosum. The stratum granulosum and corneum were negative. Melanocytes showed faint to occasional moderate staining Staining diminished to blush to faint within keratinocytes along the infundibulum and within the hair shaft. Strong staining was seen within matrix keratinocytes and melanocytes of the hair bulb. Sebaceous glands were negative to faint and eccrine sweat glands were faint to moderate. Adipocytes were negative. Within the hair follicle, the inner root sheath was faint to occasionally moderate, and the outer root sheath was negative to blush positive.

Additional samples were evaluated from patients having skin conditions or scalp with alopecia.

Sample 1: This sample of scalp was obtained at surgery from a 40-year-old female. As shown in FIG. 15, the epidermis showed strong nuclear staining of the stratum basalis, and patchy moderate to strong cytoplasmic and nuclear staining of the upper stratum spinosum and occasionally the stratum granulosum, with negative staining of the corneum. Melanocytes also showed occasional moderate to strong staining A strong immune response is shown in dermis which is infiltrated by lymphocytes and neutrophils that were moderately positive, and reactive fibroblasts that were moderately to strongly positive. Reactive capillaries were also faintly to occasionally moderate. Accumulations of macrophages were strongly positive. The ongoing severe inflammatory response is shown in a single hair root which reveals strong staining of the cells of the root, as shown in FIG. 16, and strong staining of reactive fibroblasts, the exudate, and macrophages surrounding the root. Adjacent eccrine glands also showed strong nuclear and cytoplasmic staining, with staining of secretory material in the lumen. Compared to normal skin, the sample of alopecia greata showed increased staining within cells in the epidermis, increased staining of lymphocytes, neutrophils, melanocytes, macrophages, and strong staining of reactive fibroblasts.

Sample 2: This sample of scalp was obtained at surgery of a female patient of unknown age. The epidermis showed moderate staining of the stratum basalis and stratum spinosum, with strong staining of melanocytes. Hair follicles showed moderate staining along the length of the hair, including the bulb. The hair root was also moderately positive for both nuclear and cytoplasmic staining, and surrounding fibroblasts were moderately positive. The lymphoplasmacytic inflammatory infiltrate was moderately positive. Eccrine sweat glands showed moderate nuclear staining and faint to moderate cytoplasmic staining Compared to normal skin, this sample showed slightly increased staining of epidermis, lymphocytes, melanocytes, hair follicles, and reactive fibroblasts surrounding hair follicles.

These results demonstrate the presence of ETT in skin and hair disease, the intimate involvement with the immune response and the role of L-Ergothioneine as a cellular protectant and potent antioxidant, as well as an essential bionutrient needed for cell proliferation and differentiation, according to the methods of use disclosed by the present invention. The targeted need for Ergothioneine in stem and adult tissue cells having the ETT provides further support for the methods of targeted nutrition disclosed herein through the use of medical foods and/or beverages as well as pharmaceutical and/or cosmetic compositions delivering Ergothioneine according to the invention.

1. A method of treatment for improved skin, hair and/or nail growth and maintenance of normal hair and skin colors in mammals comprising:
administering to said mammal in need of treatment thereof a source of Ergothioneine and Vitamin D; and
controlling or neutralizing free radical damage to protect against oxidative skin damage in said mammal, wherein said oxidative damage is a condition or disease state associated with decreased telomere length, decreased levels of ergothioneine, glutathione and/or Vitamin D, inflammation, oxidative stress, free radicals and/or damage to skin, hair and/or nail cells in said mammal
wherein upon administration of the same, survivability of said mammal is increased and/or progression of the disease state is decreased when compared to a mammal with such disease state without such treatment.

2. The method of claim 1 where said Ergothioneine and Vitamin D are delivered via ETT to a hair follicle bulb stem cell and bulge epithelial stem cell and result in the generation of new stem cells, new hair follicles and/or new hair shafts from dormant follicles.

3. The method of claim 1 wherein said Vitamin D is Vitamin D₂ and/or Vitamin D₃.

4. The method of claim 1 further comprising obtaining a source of Ergothioneine from any natural, extracted and/or synthesized source, including for example any whole food and/or bacteria source.

5. (canceled)

6. The method of claim 1 wherein said source of Ergothioneine and Vitamin D is a naturally enhanced, extracted and/or synthesized filamentous fungi, tissue, substrate, spent substrate or component thereof, and wherein said filamentous fungi is a mushroom of a species selected from the group consisting of: Coprinus, Agrocybe, Hypholoma, Hypsizygus, Pholiota, Pleurotus, Stropharia, Ganoderma, Grifola, Trametes, Hericium, Tramella, Psilocybe, Agaricus, Phytophthora achlya, Flammulina, Melanoleuca, Agrocybe, Morchella, Mastigomycotina, Auricularia, Gymnopilus, Mycena, Boletus, Gyromitra, Pholiota, Calvatia, Kuegneromyces, Phylacteria, Cantharellus, Lactarius, Pleurotus, Clitocybe, Lentinula (Lentinus), Stropharia, Coprinus, Lepiota, Tuber, Tremella, Drosophia, Leucocoprinus, Tricholoma, Dryphila, Marasmius, and Volvariella, and wherein said mushroom is enriched by pulsed UV irradiation without changing said mushroom's Ergothioneine content.

7. (canceled)

8. The method of claim 6 wherein the mushroom species is selected from the group consisting of Agaricus bisporus, Agaricus blazei, Lentinula edodes, Pleurotus ostreatus, and Pleurotus eryngyi.

9. The method of claim 8 wherein said fungi is in powder form.

10. The method of claim 3 wherein said Vitamin D₂ content is increased to about 800% of the daily recommended value of Vitamin D.

11. The method of claim 1 wherein said source of Ergothioneine and Vitamin D is a naturally enhanced, extracted and/or synthesized non-filamentous fungi, tissue, substrate, spent substrate or component thereof.

12. The method of claim 1 wherein the source of Ergothioneine and/or Vitamin D is a eukaryotic microorganism.

13. The method of claim 12 wherein the source of Ergothioneine and/or Vitamin D is the non-filamentous fungi yeast.

14. The method of claim 1, wherein the administration is oral, rectal, topical, transdermal buccal or sublingual, nasal, transdermal or parenteral routes and the like.

15-24. (canceled)

25. A method of treating alopecia greata, psoriasis and other forms of hair, nail and/or skin loss and/or other damage in a patient comprising:
identifying stem cells within the hair, skin and/or nails of a patient, wherein said identification uses an antibody, and
providing a source of Ergothioneine and Vitamin D for said treatment, wherein the Ergothioneine and Vitamin D stimulate stem cell proliferation and maintain the stem cells in a more viable state.

26. The method of claim 25 wherein the use of stem cells and an antibody allow identification and selection of differences in skin, nail and/or hair conditions, and further comprising at least one of the following steps: isolating the stem cells, culturing the stem cells, freezing the stem cells, wherein freezing includes the use of an extender prepared with Ergothioneine and/or Vitamin D.

27. The method of claim 25 wherein the source of Ergothioneine is a serum comprising an extracted source of Ergothioneine, Vitamin D and an additional ingredient selected from the group consisting of Vitamin D2, chitin glucans, stem cells and combinations of the same, wherein the serum is provided in the form of a cream, lotion or other topical application applied to the hair, skin and/or nails of a patient in need thereof.

28-29. (canceled)

30. A method of diagnosis comprising:
providing an antibody for the detection of ergothioneine transporter (ETT) in a patient; and
determining the deficiency, absence and/or overexpression of ETT in said patient,
wherein said detection of ETT overexpression is a diagnostic measurement for the need for supplementation of ergothioneine, and
wherein the stem cells start producing ETT and/or reverse the overexpression of ETT as a result of the administration of the Ergothioneine and/or Vitamin D.

31. (canceled)

32. The method of claim 30 wherein the overexpression or under expression of ETT allows identification of new cellular genomic targets and the ability to decode these skin targets to aid in skin repair and treat skin aging, and further comprising at least one of the following steps: comparing skin conditions and/or diseases, administering to said patient an effective amount of Ergothioneine and Vitamin D.

33-35. (canceled)

36. A method of donating electrons to maintain the body's zeta electric potential for normal skin healing, repair, and/or aiding in adherence of grafted skin used in surgical repair comprising:
providing a source of Ergothioneine and Vitamin D, wherein said Ergothioneine acts as a potent electron donor to maintain the skin's zeta electric potential,
wherein said Ergothioneine is a natural and/or synthetic source of Ergothioneine.

37. (canceled)

38. The method of claim 36 wherein Ergothioneine and Vitamin D is administered in an oral and/or topical form to deliver biological levels of the compounds, and wherein said delivery of Ergothioneine and Vitamin D aids the body's natural electric ability to rejuvenate, repair and renew skin.

39. The method of claim 38 wherein said delivery of Ergothioneine and Vitamin D aids the body's natural electric ability to rejuvenate, repair and renew skin.

40-43. (canceled)
